(19) United States
(12) Patent Application Publication
(10) Pub. No.: US 2021/0277275 A1
(43) Pub. Date: Sep. 9, 2021

(54) CROSSLINKED COATINGS

(71) Applicants: David M. STEVENS, Nashville, TN (US); Timothy T. RUCKH, Nashville, TN (US); C. Eric ELMQUIST, Nashville, TN (US); Lu TIAN, Nashville, TN (US); Pendant Biosciences, Nashville, TN (US)

(72) Inventors: David M. STEVENS, Frederick, MD (US); Timothy T. RUCKH, Mountain View, CA (US); Carl Eric ELMQUIST, Franklin, TN (US); Lu TIAN, Nashville, TN (US)

(21) Appl. No.: 16/467,189

(22) PCT Filed: Dec. 7, 2017
(86) PCT No.: PCT/US17/65148

§ 371 Date: Jun. 6, 2019

Related U.S. Application Data
(60) Provisional application No. 62/431,392, filed on Dec. 7, 2016.
(60) Provisional application No. 62/476,127, filed on Mar. 24, 2017.

(51) Int. Cl. C09D 143/02; A61L 29/08; A61L 27/34

(52) U.S. Cl. CPC C09D 143/02; A61L 2400/10; A61L 27/34; A61L 29/085

(57) ABSTRACT

The present disclosure is directed to polymeric coatings. The coatings can include a substrate-coordinating functionality as well as additional functionality for interacting with the surrounding environment. For example, the coatings can be functionalized for a variety of applications such as imparting antimicrobial properties on a substrate such as an implant; drug delivery; or as an adhesive layer between a substrate and an additional coating.

### CROSS-REFERENCE TO RELATED APPLICATIONS

This application claims the benefit of priority of U.S. Provisional Application No. 62/431,392, filed Dec. 7, 2016 and U.S. Provisional Application No. 62/476,127, filed Mar. 24, 2017, both of which are incorporated herein by reference.

The following references are also incorporated by reference herein in their entirety: U.S. Ser. No. 14/399,920, filed May 8, 2013 and now patented as U.S. Pat. No. 9,580,548; U.S. Ser. No. 11/844,353, filed Aug. 23, 2007 and now patented as U.S. Pat. No. 8,969,622; U.S. Ser. No. 12/651,710 filed Jan. 5, 2008 and currently pending; U.S. Ser. No. 12/265,701 filed Nov. 5, 2008 and now patented as U.S. Pat. No. 7,935,782; U.S. Ser. No. 13/321,897, filed May 21, 2010 and now patented as U.S. Pat. No. 9,198,985; U.S. Ser. No. 13/919,916 filed Jun. 17, 2013 and now patented as U.S. Pat. No. 9,161,983; U.S. Ser. No. 15/289,005 filed Oct. 7, 2016 and currently pending; and U.S. Ser. No. 14/605,602 filed Jan. 26, 2015 and now patented as U.S. Pat. No. 9,745,419.

### FIELD OF THE DISCLOSURE

The present disclosure is directed to polymeric coatings. The coatings can include a substrate-coordinating functionality as well as additional functionality for interacting with the surrounding environment. For example, the coatings can be functionalized for a variety of applications such as imparting antimicrobial properties on a substrate such as a medical implant; drug delivery; or as an adhesive layer between the substrate and an additional coating.

### BACKGROUND OF THE DISCLOSURE

Medical implants such as surgical implants and catheters can be susceptible to biofouling. There is a need for coatings for substrates such as medical devices and implants that can reduce the biofouling.

Hydrophilic coatings or lubricious coatings may not adhere well to medical devices and medical implants. There is a need for coatings that function as a tying layer or primer layer for bonding for hydrophilic coatings or lubricious coatings to substrates such as medical devices and medical implants.

### SUMMARY OF THE DISCLOSURE

In some embodiments, the present disclosure provides coatings that can be applied to a variety of substrates such as medical devices and medical implants. The disclosed coatings are equipped with a substrate-coordinating group such as a diol, monophosphonate, or bisphosphonate that allows the coatings to interact with a substrate surface (e.g., without needing to add any additional energy or form covalent bonds with the substrate). The coatings can help prevent biofouling and other unwanted effects of the devices and/or implants. In some cases, the coatings can have antimicrobial activities. The coatings can also be functionalized to have additional therapeutic and/or antiseptic agents. In some cases, the coatings can be used as a glue or adhesive to adhere a further layer such as a lubricious coating to the substrate.

In one aspect, the present disclosure provides a polymeric coating for a substrate, the coating comprising:

a polymeric backbone;

a substrate-coordinating group; and

a reactive functional group.

In another aspect, the present disclosure provides a method of preparing a polymeric coating for a substrate, the method comprising:
(i) preparing a polymeric backbone;
(ii) functionalizing the polymeric backbone with a substrate-coordinating group to create a functionalized polymer; and
(iii) contacting the substrate with the functionalized polymer.

In some embodiments, the resulting polymer backbones within the coating can be oriented substantially parallel to the surface of the substrate.

In another aspect, the present disclosure provides a method of preparing a polymeric coating for a substrate, the method comprising:
(i) attaching an initiator to the surface of the substrate; and
(ii) polymerizing a polymer backbone from the attached initiator.

In some embodiments, the above-aspect optionally comprises further crosslinking the polymer backbone after step (ii). In some embodiments, the resulting polymer backbones within the coating can be oriented substantially perpendicular to the surface of the substrate.

In some embodiments of any of the above-aspects, the polymeric backbone comprises a polyglycidol or a polyester such as polyvalerolactone, polyglycolic acid, polylactic acid, or co-polymers thereof. In some embodiments, the polyglycidol backbone comprises a polyallyl glycidyl ether-polyglycidol copolymer. In some embodiments, the polyglycidol backbone is linear, branched, or hyperbranched. In some embodiments, the polyglycidol backbone is branched.

In some embodiments, the polyester backbone comprises a poly(valerolactone) backbone (e.g., a polyallylvalerolactone-polyvalerolactone copolymer backbone). In some embodiments, the poly(valerolactone) backbone can comprise poly(epoxy-δ-valerolactone) (evl); poly(α-allyl-δ-valerolactone) (avl); poly(2-oxepane-1,5-dione) (opd); poly(α-propargyl-δ-valerolactone) (ppvl); or combinations thereof.

In some embodiments, the substrate is metal. In some embodiments, the substrate-coordinating group is a metal-coordinating group. In some embodiments, the metal-coordinating group is a monophosphonate group, a bisphosphonate group, a diol, a thiol, an amine, a pyrrol-containing group, or a catechol. In some embodiments, the bisphosphonate group is selected from the group consisting of: alendronate; risendronate; etidronate; clodronate; tiludronate; pamidronate; neridronate; olpadronate; ibandronate; and zoledronate. In some embodiments, the diol is selected from the group consisting of: ethylene glycol; and propylene glycol.

In some embodiments, the reactive functional group is an alkene, an alkyne, or an epoxide. In some embodiments, the coating further comprises a binding agent.

In some embodiments, the polymeric coatings comprise a polymer of Formula I:

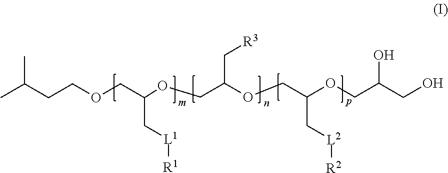

wherein:

L¹ is independently, at each occurrence, —(CR^{1A}R^{1B})_{q}—, —O(CR^{1A}R^{1B})_{q}—, —(CR^{1A}R^{1B})O_{q}—, —(CR^{1A}R^{1B})C(O)O_{q}— or —OC(O)(CR^{1A}R^{1B})_{q}—;

L² is independently, at each occurrence:

—(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;
—O(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;
—C(O)(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—; or
—OC(O)(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;

R¹ is independently, at each occurrence, —C₁-C₆ alkyl, —C₁-C₆ alkenyl, —C₁-C₆ alkynyl, —CH(O)CH₂, or —OH, wherein each alkyl, alkenyl, or alkynyl is optionally substituted with a drug, a conjugating group, a -PEG₁₋₆ bonded drug, or a -PEG₁₋₆ bonded conjugating group;

R^{1A} and R^{1B} are each independently, at each occurrence, —H —OH, or —NH₂;

R² is independently, at each occurrence, —CR^{2C}R^{2D}P(O)(OH)₂; —CR^{2C}(P(O)(OH)₂)₂; —C(P(O)(OH₂)₃, —CO₂H, —C₆(R^{2A})₃(OH)₂, or —CR^{2A}R^{2B}CR^{2B}R^{2C}R^{2D};

R^{2A} and R^{2B} are each independently, at each occurrence, —H —OH, or —NH₂;

R^{2C} and R^{2D} are each independently, at each occurrence, —H, —OH or —NH₂;

R³ is independently, at each occurrence, —C₁-C₆ alkenyl, —C₁-C₆ alkynyl, —C(O)C₁-C₆ alkyl, —C(O)C₁-C₆ alkenyl, —C(O)C₁-C₆ alkynyl, —OH, —OC₁-C₆ alkyl, —OC₁-C₆ alkenyl, —OC₁-C₆ alkynyl, —OC(O)C₁-C₆ alkyl, —OC(O)C₁-C₆ alkenyl, —OC(O)C₁-C₆ alkynyl, wherein each alkyl, alkenyl, or alkynyl is optionally substituted with a drug, a conjugating group, a -PEG₁₋₆ bonded drug, or a -PEG₁₋₆ bonded conjugating group;

m is independently an integer between 1 and 10,000;

n is independently an integer between 1 and 10,000;

p is independently an integer between 1 and 10,000;

q is independently, at each occurrence, an integer between 0 and 6; and

t is independently, at each occurrence, 0 or 1.

In some embodiments, the polymeric coating is a polymer of Formula I-A(1):

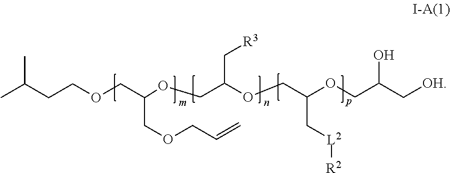

In some embodiments, the polymeric coating is a polymer of Formula I-A(2):

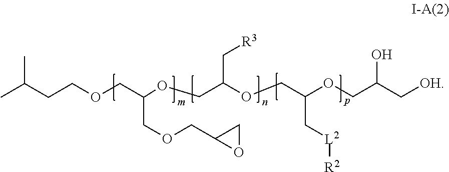

In some embodiments, the polymeric coating is a polymer of Formula I-A(3):

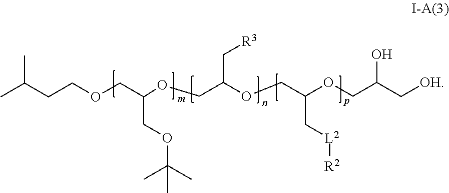

In some embodiments, the polymeric coating is a polymer of Formula I-B(1):

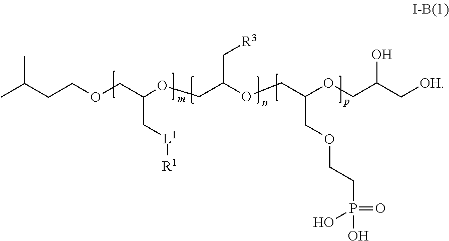

In some embodiments, the polymeric coating is a polymer of Formula I-B(2):

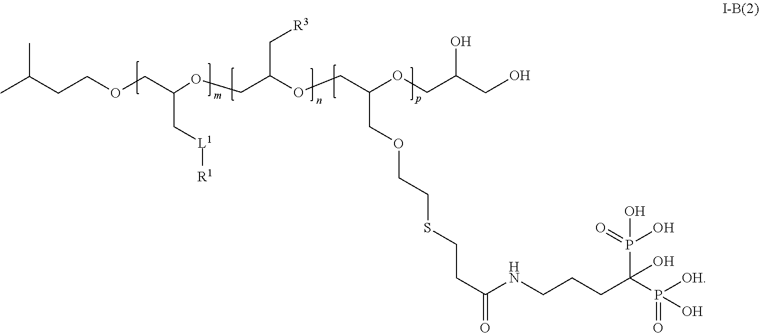

In some embodiments, the polymeric coating is a polymer of Formula I-B(3):

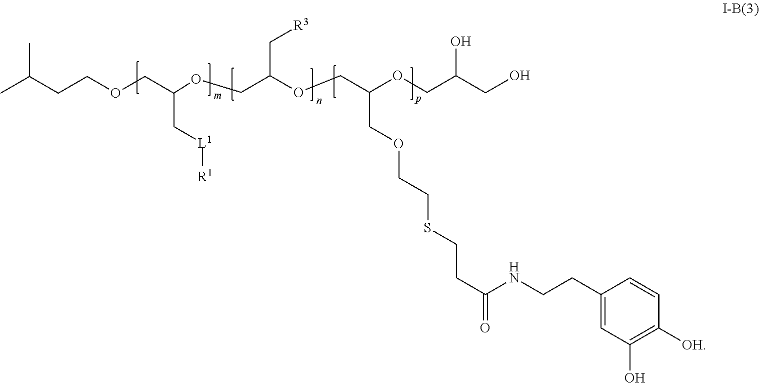

In some embodiments, the polymeric coating is a polymer of Formula I-C(1):

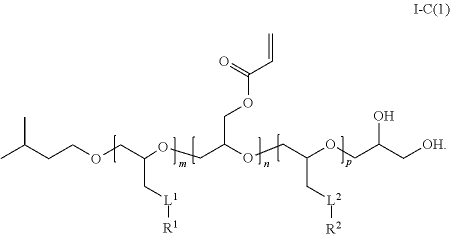

In some embodiments, the polymeric coating is a polymer of Formula I-C(2):

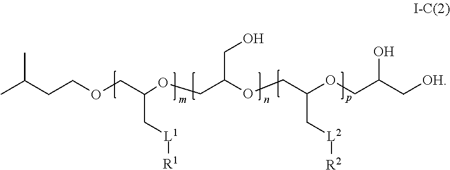

In some embodiments, the polymeric coating is a polymer of the Formula I-C(3):

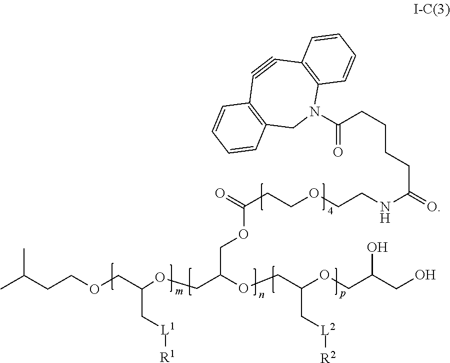

In some embodiments, the polymeric coating is a polymer of the Formula I-C(4):

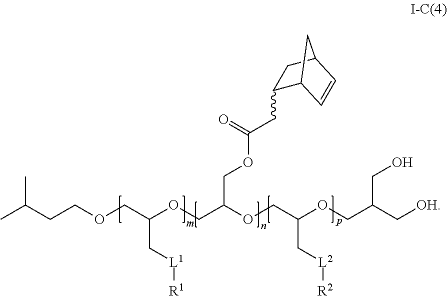

In some embodiments, the polymeric coating is a polymer selected from the group consisting of:

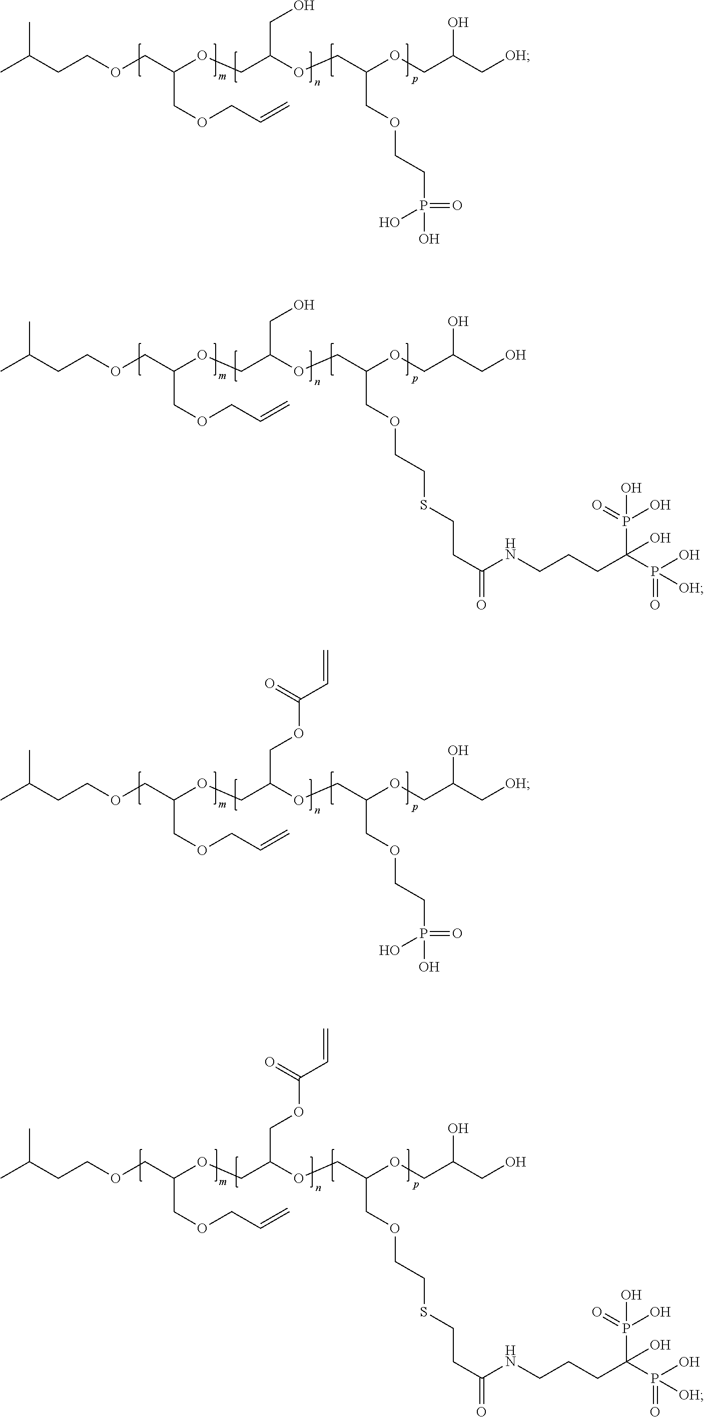

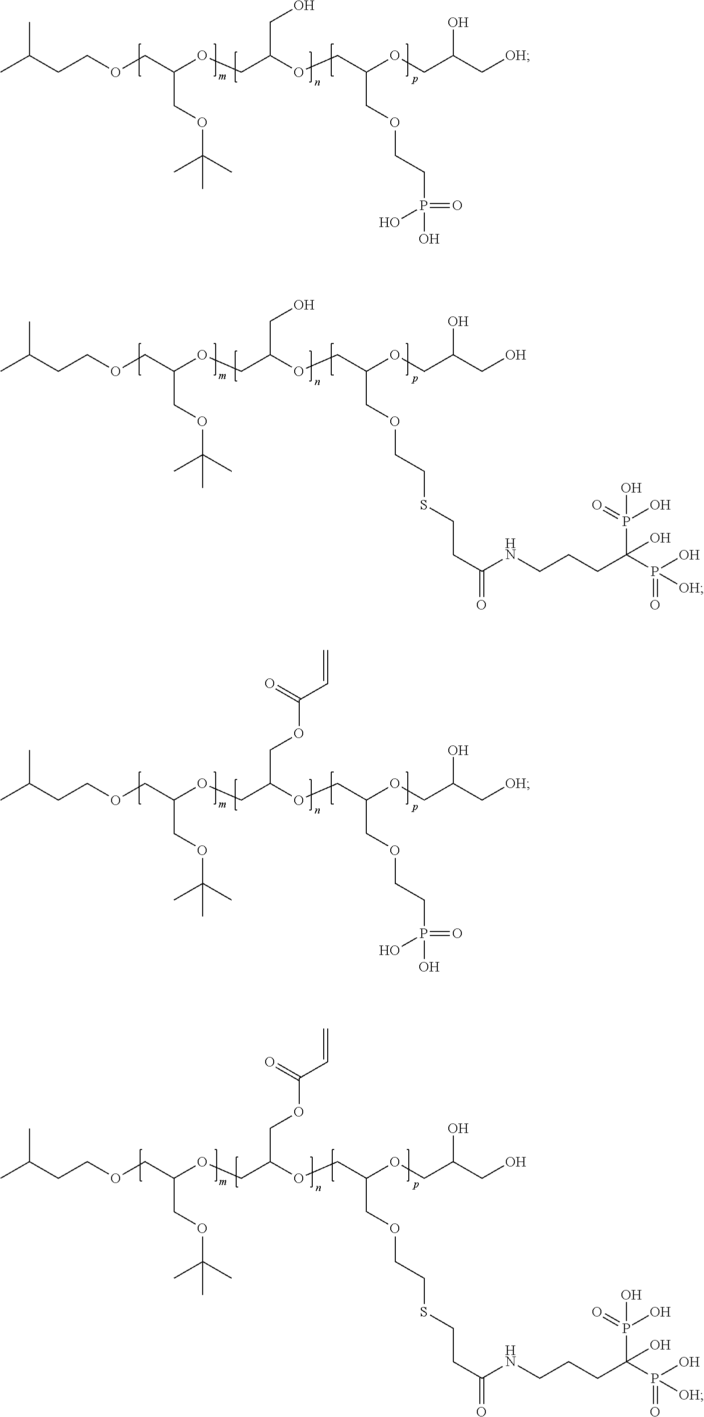

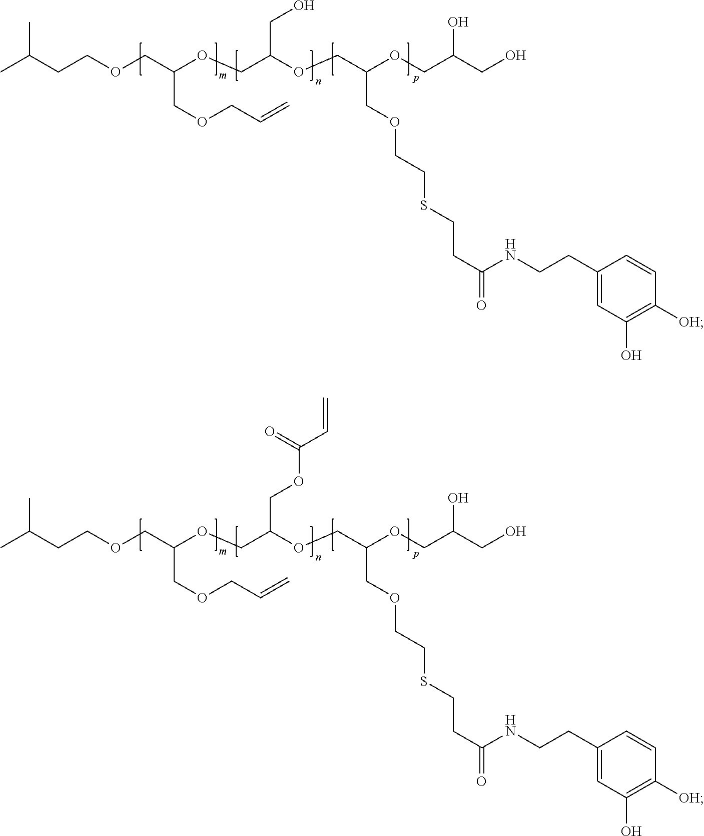

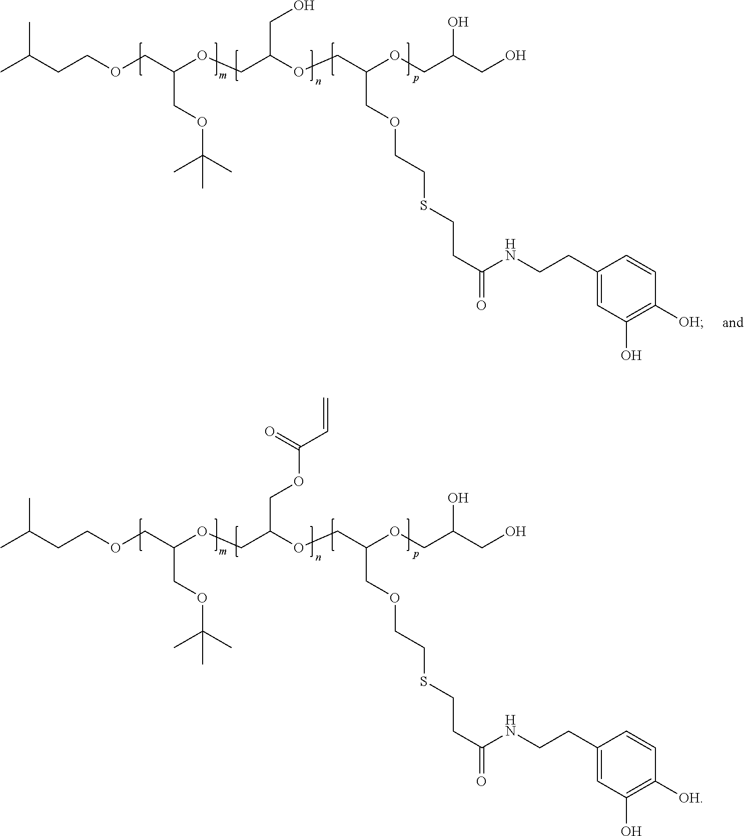

In some embodiments, the polymeric coating is a polymer of Formula II:

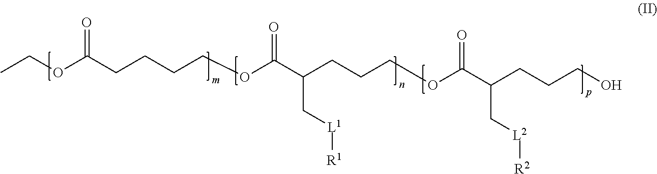

wherein:

L¹ is independently, at each occurrence, —(CR^{1A}R^{1B})_{q}—, —O(CR^{1A}R^{1B})_{q}—, —(CR^{1A}R^{1B})O_{q}—, —(CR^{1A}R^{1B})C(O)O_{q}—, —(CR^{1A}R^{1B})OC(O)_{q}—, or —OC(O)(CR^{1A}R^{1B})_{q}—;

L² is independently, at each occurrence:

—(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;
—O(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;
—C(O)(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—; or
—OC(O)(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;

R¹ is independently, at each occurrence, —C₁-C₆ alkyl, —C₁-C₆ alkenyl, —C₁-C₆ alkynyl, —CH(O)CH₂, or —OH, wherein each alkyl, alkenyl, or alkynyl is optionally substituted with a drug, a conjugating group, a -PEG₁₋₆ bonded drug, or a -PEG₁₋₆ bonded conjugating group;

R^{1A} and R^{1B} are each independently, at each occurrence, —H —OH, or —NH₂;

R² is independently, at each occurrence, —CR^{2C}R^{2D}P(O)(OH)₂; —CR^{2C}(P(O)(OH)₂)₂; —C(P(O)(OH₂)₃, —CO₂H, —C₆(R^{2A})₃(OH)₂, or —CR^{2A}R^{2B}CR^{2B}R^{2C}R^{2D};

R^{2A} and R^{2B} are each independently, at each occurrence, —H —OH, or —NH₂;

R^{2C} and R^{2D} are each independently, at each occurrence, —H, —OH or —NH₂;

m is independently an integer between 1 and 10,000;

n is independently an integer between 1 and 10,000;

p is independently an integer between 1 and 10,000;

q is independently, at each occurrence, an integer between 0 and 6; and

t is independently, at each occurrence, 0 or 1.

In some embodiments, the polymeric coating is a polymer of Formula II-A(1):

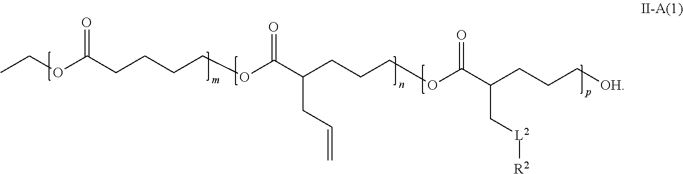

In some embodiments, the polymeric coating is a polymer of Formula II-A(2):

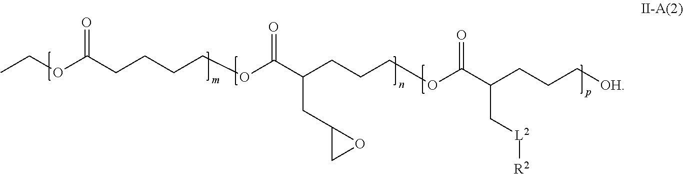

In some embodiments, the polymeric coating is a polymer of the Formula II-A(3):

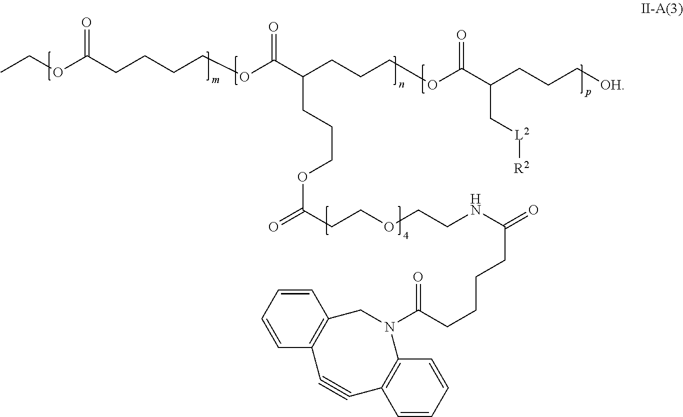

In some embodiments, the polymeric coating is a polymer of the Formula II-A(4):

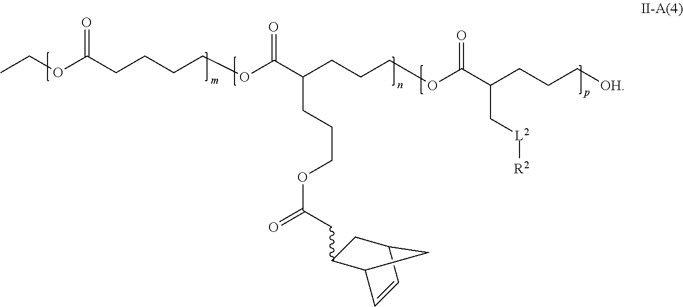

In some embodiments, the polymeric coating is a polymer of Formula II-B(1):

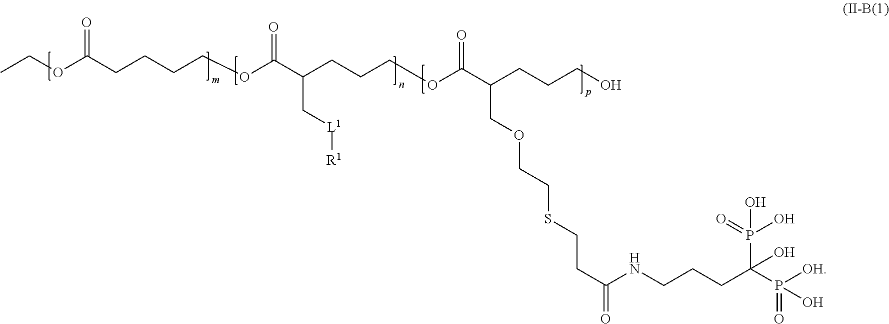

In some embodiments, the polymeric coating is a polymer of Formula II-B(2):

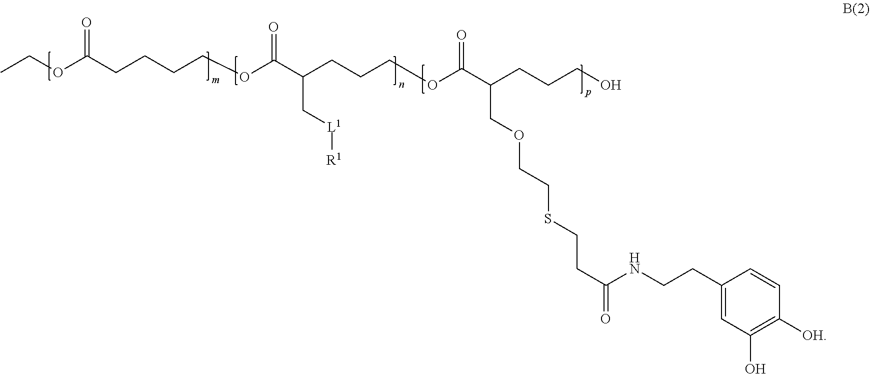

In some embodiments, the polymeric coating is a polymer selected from the group consisting of:

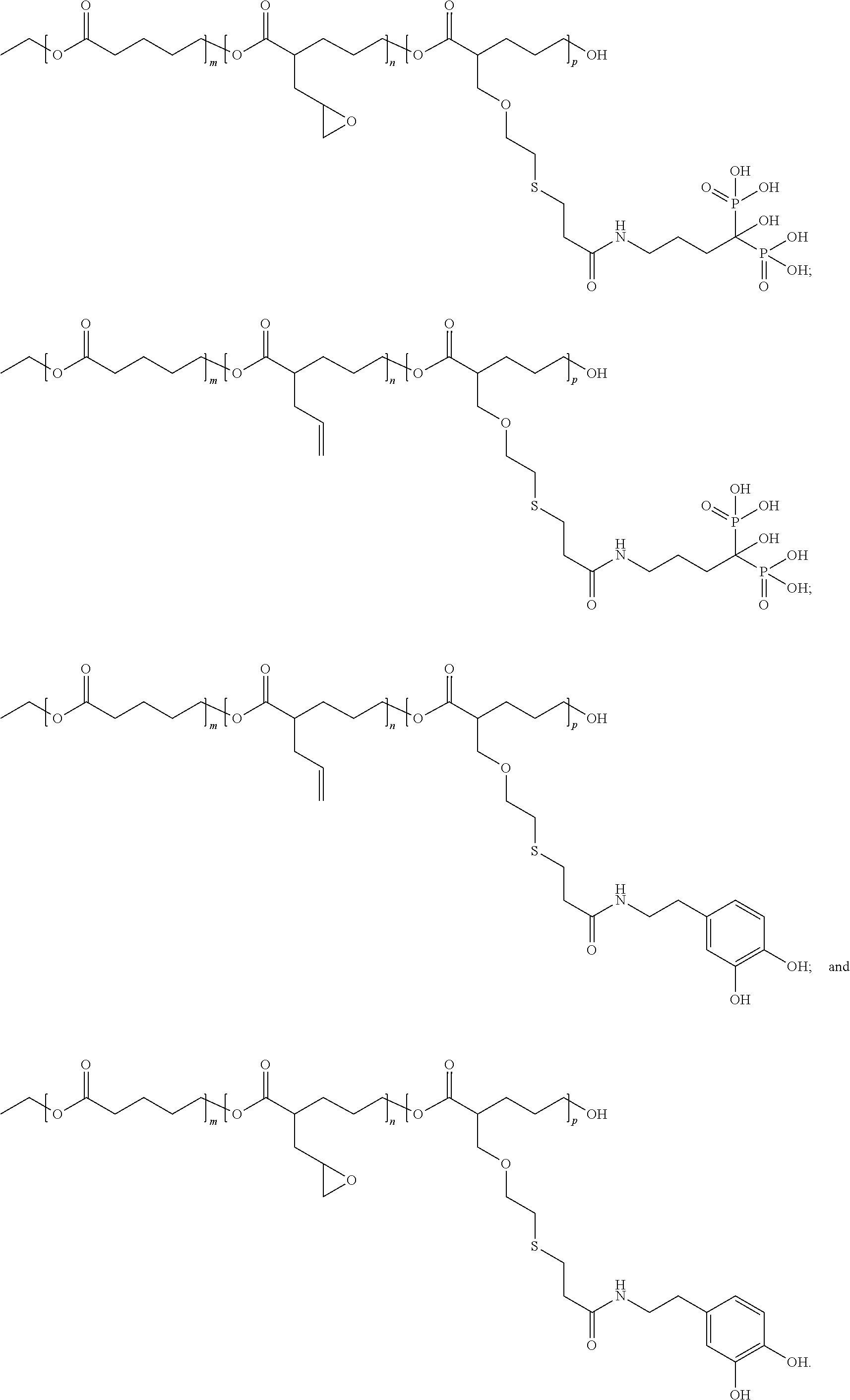

In some embodiments, the polymeric backbone is crosslinked. In some embodiments, crosslinker is polyethylene glycol. In some embodiments, the coating is biodegradable. In some embodiments, the coating is functionalized with a drug. In some embodiments, the coating is functionalized with an antibody. In some embodiments, the coating is functionalized with a further lubricious coating. In some embodiments, contacting the substrate with the functionalized polymer comprises dip-coating, spray-coating, or flow-coating the substrate in the functionalized polymer.

In another aspect, the present disclosure provides a coated substrate, wherein the coating comprises:

a polymeric backbone;

a substrate-coordinating group; and

a reactive functional group.

In some embodiments, the substrate comprises steel, titanium, nickel-titanium alloy, or cobalt-chromium alloy. In some embodiments, the substrate is a medical device. The coating can be any of the polymeric coatings described herein.

### DETAILED DESCRIPTION OF THE DISCLOSURE

### Definitions

The articles “a” and “an” are used in this disclosure to refer to one or more than one (e.g., to at least one) of the grammatical object of the article. By way of example, “an element” means one element or more than one element.

The term “and/or” is used in this disclosure to mean either “and” or “or” unless indicated otherwise.

As used herein, the term “vl” or “VL” is understood to mean valerolactone. As used herein “PVL” is understood to mean poly valerolactone.

As used herein the term “avl” or “AVL” is used to mean allyl valerolactone. As used herein, the term “PAVL” is understood to mean polyallylvalerolactone.

Accordingly, the term “poly(avl-vl)” or “poly(vl-avl)” is understood to mean a copolymer comprising allyl valerolactone and valerolactone. This term includes all embodiments of the copolymer including a block copolymer and a random copolymer.

As used herein, the term “epoxy” is understood to mean an epoxide. An unsubstituted epoxide can be abbreviated as “—CH(O)CH₂” or as

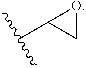 [SMILES: *C1CO1]

In some embodiments, an epoxide group can be substituted (e.g., with one or more alkyl, alkenyl, or alkynyl groups).

“Alkyl” refers to a straight or branched chain saturated hydrocarbon. C₁-C₆ alkyl groups contain 1 to 6 carbon atoms. Examples of a C₁-C₆ alkyl group include, but are not limited to, methyl, ethyl, propyl, butyl, pentyl, isopropyl, isobutyl, sec-butyl and tert-butyl, isopentyl and neopentyl.

The term “alkenyl” means an aliphatic hydrocarbon group containing a carbon-carbon double bond and which may be straight or branched having about 2 to about 6 carbon atoms in the chain. Alkenyl groups can have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. A C₂-C₆ alkenyl group is an alkenyl group containing between 2 and 6 carbon atoms.

The term “alkynyl” means an aliphatic hydrocarbon group containing a carbon-carbon triple bond and which may be straight or branched having about 2 to about 6 carbon atoms in the chain. Alkynyl groups can have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl. A C₂-C₆ alkynyl group is an alkynyl group containing between 2 and 6 carbon atoms.

An “ester” is a chemical linkage defined as

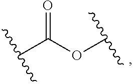 [SMILES: *OC(*)=O]

wherein “R” and “R′” are each carbon-based substituents. As used herein, a “polyester” is a polymer that contain the ester functional group in its main chain.

As used herein, “PEG” is understood to mean polyethylene glycol. PEG is a polymer comprising repeating units of —OCH₂CH₂—. In some embodiments, PEG can be used to connect a reactive functional group (e.g., a conjugating group or a drug) to a polymer backbone. As used herein, a PEG₁₋₆ group comprises between one and six repeating units of —(OCH₂CH₂)ₙ—. As used herein, a PEG linker can be oriented to connect to a functional group by a carbon atom or an oxygen atom. For example, a PEG chain can be oriented as —(OCH₂CH₂)ₙ— or as —(CH₂CH₂O)ₙ—. In some embodiments, when a PEG is used to attach a functional group to a polymer backbone, the point of attachment of the PEG can include a heteroatom other than oxygen to as a connecting point between the PEG and the functional group. For instance, when dibenzocyclooctyne-PEG4-acid is attached as a functional group to a polymer backbone, it can be condensed via a nitrogen atom to form an amide linkage with the PEG linker. In other words, the PEG linker can include an —NH— group in place of an —O— group to form an amide bond (i.e., instead of an ester) with the functional group.

### Description of the Compounds and Synthesis Thereof

The present disclosure teaches polymeric backbones that can be used to coat a substrate such as a surface for a medical device or implant. In some embodiments, the polymers of the present disclosure are at least bifunctional. More specifically, in some embodiments, the polymers of the present disclosure can have a substrate-coordinating group that can be oriented toward the substrate and that can interact with the substrate. In some embodiments, the polymers can also have a reactive functional group in addition to the substrate-coordinating group. In some embodiments, the reactive functional group can be oriented away from the substrate-coordinating group and can interact with the environment outside the substrate. In some embodiments, the reactive functional group is functionalized to crosslink the polymers to form a coating. In some embodiments, the reactive functional group is functionalized to bind to a different polymer or chemical substance. In some embodiments, the reactive functional group is functionalized to bind to a drug.

### Polymer Backbones

The present disclosure contemplates the use of multiple polymer backbones for use in the preparations of the polymeric coatings described herein. As used herein, the term “polymer backbone” refers to a chemically inert polymer comprising a plurality of monomers. Certain features of the disclosure (e.g., a substrate-coordinating group, a reactive functionality) can be bonded to the polymer backbone. In some embodiments, the molecular weight of the polymers herein (e.g. functionalized polymers) can be between about 3,000 and about 100,000 g/mol (e.g., about 20,000 g/mol or below).

Polymer backbones that may be used to prepare a polymeric coating of the present disclosure include, but are not limited to, poly(N-acetylglucosamine) (Chitin), Chitosan, poly(hydroxybutyrate), poly(hydroxybutyrate-co-valerate), polyorthoester, polyanhydride, polyester amide, poly(glycolic acid-co-trimethylene carbonate), copoly(ether-esters) (e.g. PEO/PLA), polyphosphazenes, biomolecules (e.g., fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrilestyrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose, poly(lactic acid). Polymers that can be used includes graft copolymers, and block copolymers, such as AB block copolymers (“diblock-copolymers”) or ABA block-copolymers (“triblock-copolymers”), or mixtures thereof.

In some embodiments, polymerization of the polymer backbones occurs in the solid-state. In such an arrangement, the metal surface may be oxidized using an oxidizing agent and optionally coating with a silanizing step to stably install polymerization initiator sites on the implant surface. For example, the polymerization of the polymer backbone can occur in the solid state as set forth in U.S. Pat. No. 7,160,592 to Rypacek, the contents of which are incorporated by reference in their entirety.

### Polyester Backbones

In some embodiments, the polymer backbone can be or include a polyester or functionalized polyester. As used herein, a polyester is a polymer comprising repeating ester units in the polymer backbone. For example, polyesters that can be used in accordance with the present disclosure include, e.g., poly(valerolactone) (PVL), including poly(allylvalerolactone) (PAVL); poly(caprolactone) (PCL); poly(lactic acid) (PLA); poly(lactic-co-glycolic acid) (PLGA). In some embodiments, the polymer backbone can include a combination or mixture of any polyesters or other polymers, including those described above. In some embodiments, a polyester backbone of the present disclosure can be used in combination (e.g., as a block copolymer) with other polymers such as PEG or polyglycidol.

For example, in some embodiments, the polymer backbone comprises a copolymer (e.g., a block copolymer or random copolymer) of a functionalized monomer and a substantially inert monomer. For example, the polymer backbone can comprise both PLGA (i.e., a substantially inert monomer) and allyl lactide (i.e., a functionalized monomer). The resulting polymer backbone can be, e.g., a poly(lactic-co-glycolic acid)-co-poly(allyl lactide) copolymer. In some embodiments, co-polymerizing PLGA and allyl lactide can result in a functionalized (i.e., an allyl-functionalized) polymer backbone e.g., wherein some repeating units such as the lactic and/or glycolic acid units are substantially inert. Without wishing to be bound by theory, preparation of a copolymer comprising functionalized and non-functionalized (i.e., inert) monomers such as poly(lactic-co-glycolic acid)-co-poly(allyl lactide) copolymer is disclosed in U.S. Pat. No. 8,875,828 to Markland et al., the contents of which are hereby incorporated by reference in their entirety.

For example, a functionalized polyester comprising a functional group can be prepared starting from a cyclic dimer of the formula:

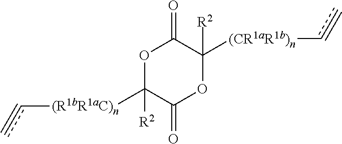

wherein n is an integer between 0 and 12;

R^{1a} and R^{1b} are each hydrogen, hydroxy, amino, thio, halogen, substituted or unsubstituted C₁-C₆ alkyl, substituted or unsubstituted C₁-C₆alkoxy, substituted or unsubstituted C₁-C₆alkylthio, substituted or unsubstituted C₁-C₆alkylamino, or substituted or unsubstituted C₁-C₆ hydroxy alkyl;

R² is hydrogen, hydroxy, amino, thio, halogen, substituted or unsubstituted C₁-C₆ alkyl, substituted or unsubstituted C₁-C₆alkoxy, substituted or unsubstituted C₁-C₆alkylthio, substituted or unsubstituted C₁-C₆ alkylamino, or substituted or unsubstituted C₁-C₆ hydroxy alkyl; and

wherein [symbol] is an optional bond.

In some embodiments, the functional group can be an allyl group, and the allyl-functionalized polymer can be prepared from a starting dimer of the formula:

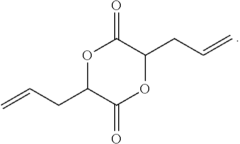

In some embodiments, a functionalized polyester (e.g., a functionalized cyclic dimer as set forth above) can be co-polymerized in the presence of a substantially inert (i.e., non-functionalized) starting material such as lactic acid, glycolic acid, caprolactone, or a combination thereof. In some embodiments, a functionalized starting material (e.g., a cyclic dimer as set forth above) can be polymerized in the presence of a dimer of lactic acid and/or glycolic acid or in the presence of caprolactone. For example, a functionalized starting material (e.g., a functionalized cyclic dimer as set forth above) can be polymerized in the presence of one or a combination of:

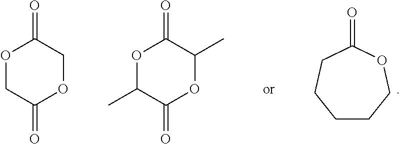

In some embodiments, a polymer backbone comprising a functionalized monomer and a substantially inert monomer can be a block copolymer (i.e., the polymer can have one or more blocks of functionalized monomer units and one or more blocks of substantially inert units). In some embodiments, the polymer backbone can be a random copolymer of a functionalized monomer and a substantially inert monomer.

In some embodiments, the polymerization reaction can be catalyzed by a catalyst. For example the polymerization catalyst can be metallic or non-metallic, including a variety of non-metallic organic catalysts. Suitable metal catalysts include zinc powder, tin powder, aluminum, magnesium and germanium, metal oxides such as tin oxide (II), antimony oxide (III), zinc oxide, aluminum oxide, magnesium oxide, titanium oxide (IV) and germanium oxide (IV), metal halides such as tin chloride (II), tin chloride (IV), tin bromide (II), tin bromide (IV), antimony fluoride (III), antimony fluoride (V), zinc oxide, magnesium chloride and aluminum chloride, sulfates such as tin sulfate (II), zinc sulfate and aluminum sulfate, carbonates such as magnesium carbonate and zinc carbonate, borates such as zinc borates, organic carboxylates such as tin acetate (II), tin octanoate (II), tin lactate (II), zinc acetate and aluminum acetate, organic sulfonates such as tin trifluoromethane sulfonate (II), zinc trifluoromethane sulfonate, magnesium trifluoromethane sulfonate, tin (II) methane sulfonate and tin (II) p-toluene sulfonate. Dibutyltin dilaurate (DBTL), Sb₂O₃, Ti(IV)_{bu}, Ti(IV)ᵢₛₒ, and others can also be used.

### Poly(valerolactone) (PVL) Polymers

In some embodiments, poly(valerolactone) can serve as the polymer backbone. In some embodiments, the poly(valerolactone) is a poly(allylvalerolactone-valerolactone) copolymer (sometimes abbreviated poly(avl-vl)). The percentage of monomers in a poly(valerolactone) backbone that have an attached allyl group can be between about 1 and 100.

As set forth in the Examples below, a poly(allylvalerolactone-valerolactone) copolymer can be prepared by, for instance, copolymerizing valerolactone and allylvalerolactone. In some embodiments, the polymerization takes place in the presence of a catalyst (e.g., tin triflate), as shown below:

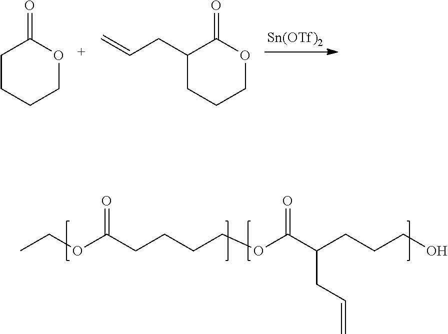

Poly(allylglycidol-glycidol) PGL Polymers and Copolymers

In some embodiments, poly(glycidol) (PGL) can serve as the polymer backbone. In some embodiments, the poly(glycidol) is a poly(allylglycidol-glycidol) copolymer. As set forth in the Examples below, a poly(allylglycidol-glycidol) copolymer can be prepared by, for instance, copolymerizing glycidol and allylglycidol. In some embodiments, the polymerization takes place in the presence of a catalyst (e.g., tin triflate).

A poly(allylglycidol-glycidol) copolymer can be prepared in several different varieties. For example, as set forth below, a poly(allylglycidol-glycidol) copolymer can be, for instance, linear, semi-branched, or hyper branched. In some embodiments, the polyglycidol backbones of the present disclosure can be used in combination (e.g., as a block copolymer) with other polymers such as PEG or polyesters.

In some embodiments, polymerization of the poly(allylglycidol-glycidol) polymer backbones can occur in the absence of metal catalysis. Such synthetic route may be considered to be “green chemistry” because the aqueous solution is not harmful to the environment. For example, polymerization can occur using 1,5,7-triazabicyclo[4.4.0]dec-δ-ene (TBD) as a catalyst. Examples of metal-free catalysis can be found in, e.g., Silvers, A. L. et al., J. Polymer Sci. Part A: Polymer Chemistry, 2012; 50:3517-3529; and Parrish, B. et al., J. Polymer Sci. Part A: Polymer Chemistry, 2002; 40:1983-1990.

In some embodiments, polymerization of the poly(allylglycidol-glycidol) polymer backbones can occur in the absence of catalysis. Such synthetic route may be considered to be “green chemistry” because the aqueous solution is not harmful to the environment. For example, the polymerization of the polymer backbone can occur in the solid state as set forth in U.S. Patent Publication 2015/0210805 to Harth, the contents of which are incorporated by reference in their entirety.

### Linear Polyglycidol

In some embodiments, liner polyglycidol can be made using an alkyl-protected glycidol monomer (e.g., allyl glycidyl ether, tert-butyl glycidyl ether) and an alcohol as an initiator (e.g., ethanol, benzyl alcohol), and subsequent removal of the alkyl protecting group (e.g., under acidic conditions). Other protected glycidol monomers such as ethoxy glycidyl ether can also be used. A general reaction scheme for the preparation of linear polyglycidol polymers is shown below:

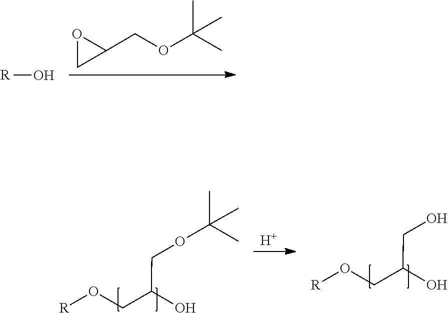

Semi-Branched Polyglycidol

In some embodiments, a semi-branched polyglycidol can be used to prepare the functionalized polymer coatings of the disclosure. For example, a semi-branched polyglycidol can be prepared in the presence of tin triflate (e.g., between about −80° C. to about 50° C.). In some embodiments, a semi-branched polyglycidol can be prepared in an aqueous buffer (e.g., about pH 3 to about pH 9) and at temperatures between about 50° C. and about 120° C. A general reaction scheme for the preparation of semi-branched polyglycidol polymers is shown below:

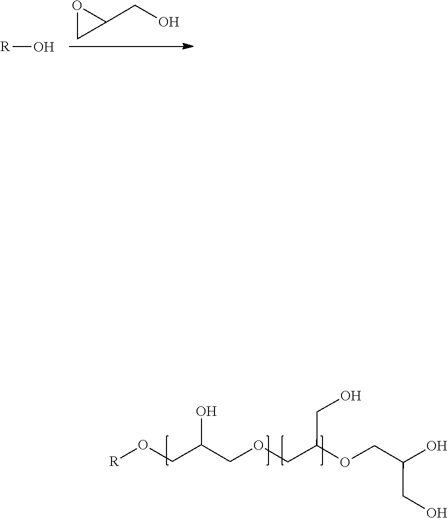

### Hyper-Branched Polyglycidol

In some embodiments, a hyper-branched polyglycidol can be used to prepare the functionalized polymer coatings of the disclosure. For example, a hyper-branched polyglycidol can be prepared in the presence of a multi-alcohol initiator. In some embodiments, a hyper-branched polyglycidol can be prepared in similar conditions (e.g., the same conditions) that are used to prepare a semi-branched polyglycidol polymer, except that di-, tri-, or multi-functional alcohols can be used. The schemes below give examples of the preparation of hyper-branched polyglycidols:

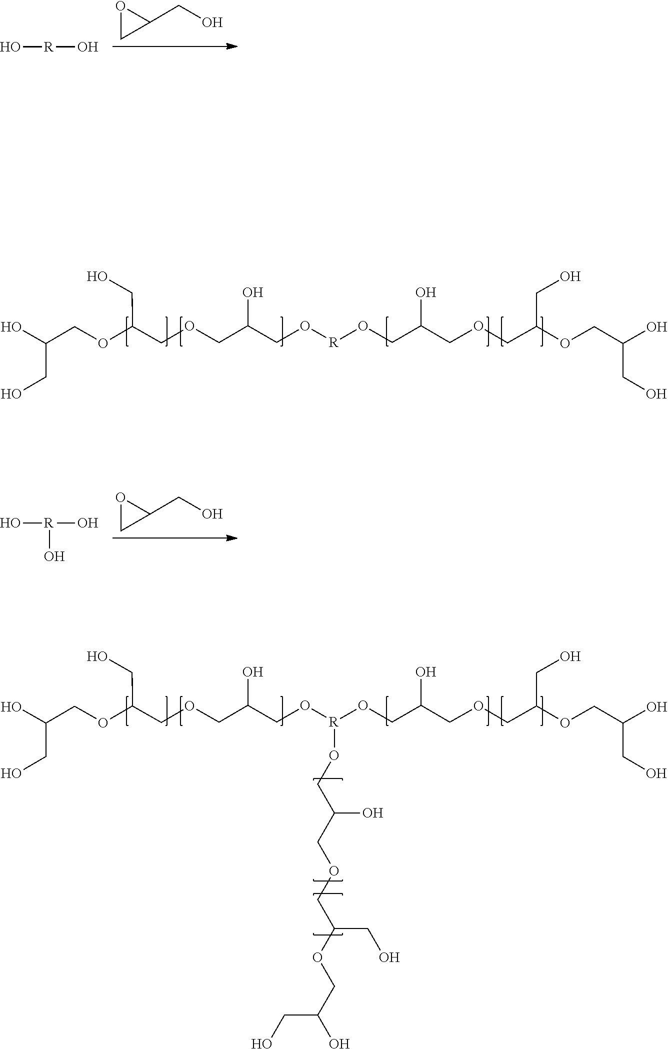

### PEG Copolymers

In some embodiments, the polymeric backbones of the present disclosure can be PEG copolymers. For example, in some embodiments, PEG can be incorporated into a polymer backbone (e.g., a polyglycidol, a polyester, or a mixture thereof) of the present disclosure. In some embodiments, the polymeric backbones of the present disclosure can be poly(allylvalerolactone-valerolactone)-PEG copolymers. In some embodiments, the polymeric backbones of the present disclosure can be poly(allylglycidol-glycidol)-PEG copolymers.

In some embodiments, PEG can be incorporated into a poly(avl-vl) polymer backbone. In some embodiments, the PEG can be a PEG diol or PEG dithiol. In some embodiments, PEG can be incorporated into a polymer backbone using a triol (e.g., 1,1,1-tris(hydroxymethyl)ethane. In some embodiments, a tin (Sn) catalyst is used to incorporate the PEG copolymer.

In some embodiments, the polymer backbone of the present disclosure is a P22 triblock copolymer such as (PVL-co-PAVL)-b-PEG-b-(PVL-co-PAVL). In some embodiments, the polymer is (PVL₃₅-co-PAVL₆)-b-PEG₈₈-b-(PVL₆-co-PAVL₃₅).

Example 3 below teaches the synthesis of a pentablock copolymer (PAVL-b-PVL-b-PEG-b-PVL-b-PAVL). As set forth in Example 3, PEG was used as an initiator to create a block copolymer with valerolactone to create PVL-b-PEG-b-PVL. Next, allylvalerolactone was added to prepare PAVL-b-PVL-b-PEG-b-PVL-b-PAVL. As set forth in Example 3, the molecular weight of the PEG was about 20,000, the target number of valerolactone (VL) repeating units was about 100, and the target number of allylvalerolactone (AVL) units was about 25.

In some embodiments, the PEG initiator can have a molecular weight of between about 1 k and about 50 k. In some embodiments, the number of VL units is between about 0 and about 500. In some embodiments, the number of AVL units is between about 0 and about 500. In some embodiments, the VL and AVL monomers are added at the same time to produce P(AVL-co-VL)-b-PEG-b-(PAVL-co-VL) triblock copolymer. In some embodiments, a multi-arm PEG initiator (i.e. 4-arm or 8-arm) is used to form branched polymers. In some embodiments, methoxy-PEG is used as the initiator to form PEG-b-PAVL-b-PVL triblock polymer.

In some embodiments, methoxy-PEG is used as the initiator to form PEG-b-P(AVL-co-VL) copolymer. In some embodiments, a mono alcohol (e.g., benzyl alcohol, methanol, ethanol, or propanol) is used as an initiator to form PAVL-b-PVL. In some embodiments, a mono alcohol (e.g., benzyl alcohol, methanol, ethanol, or propanol) is used as an initiator to form P(AVL-co-VL).

In some embodiments, caprolactone is used instead of valerolactone. For instance, alpha-allyl-caprolactone can be used instead of alpha-allyl-valerolactone. In some embodiments, caprolactone and alpha-allyl-caprolactone are used instead of valerolactone and alpha-allyl-valerolactone. In some embodiments, a metal surface is used as the initiator for a polymerization to perform solid-support polymerization.

### Substrate-Coordinating Groups

The polymer backbones of the present disclosure can be functionalized with substrate-coordinating groups. In some embodiments, the substrate can be a medical device or medical implant. The substrate can comprise a metal or polymeric (e.g., plastic) surface. In some embodiments, the substrate is glass (e.g., a glass surface). In some embodiments, the substrate is a metal surface. The metal surface can be a pure metal or can be a metal alloy. For instance, the metal surface can be steel (e.g., stainless steel), titanium, 316L steel, nickel-titanium alloy (i.e., nitinol), or cobalt-chromium alloy.

In some embodiments, the substrate-coordinating groups can be capable of forming a transient bond with a metal surface. For example, substrate-coordinating groups can include phosphates or phosphonates (e.g., etidronate clodronate, tiludronate, pamidronate, neridronate, olpadronate, alendronate, ibandronate, risendronate, and zoledronate). In some embodiments, the substrate-coordinating group is a catechol (e.g., a catecholamine such as dopamine or polydopamine); carboxylic acids such as aminocarboxylic acids (e.g., aspartic acid, EDTA, ethylenediamine-N,N′-diacetic acid, [(2-Aminoethyl)amino]acetic acid, and iminodiacetic acid); Crown ethers; or Calix-arenes. In some embodiments, the substrate-coordinating group can be a nitrogen-containing coordination group such as an indole (e.g., 5-(aminomethyl)indole), an imidazole (e.g., (4(5)-(Hydroxymethyl)imidazole), an aliphatic primary amine (e.g., cystamine).

In some embodiments, the substrate can be prepared to enable greater adherence of a substrate coordinating group. In an embodiment, the surface of the substrate is subjected to oxidative, photo-oxidative and/or polarizing surface treatment, for example plasma and/or corona treatment in order to improve the adherence of the polymer coating. Suitable conditions are known in the art.

In some embodiments, catechols can coordinate to metal surfaces such as iron (e.g., iron (III)) or nickel. In some embodiments, when a catechol is used as a substrate-coordinating group, the catechol can be first protected (e.g., using an acetal protecting group).

Shown below is a general scheme depicting the functionalization of a polyglycidol backbone with a monophosphonate. As depicted below, a polyglycidol (e.g., a linear polyglycidol, a branched polyglycidol or a hyperbranched polyglycidol) can be functionalized with an alkyl-protected phosphonic acid by condensing a vinyl phosphonate onto a free hydroxyl group of the polymer backbone (Step 1). The polyglycidol can be functionalized with the vinyl phosphonate using a conjugate addition (e.g., Michael-type addition). In some embodiments, the reaction can be catalyzed (e.g., with a metal alkoxide such as KO^{t}Bu). Deprotection of the phosphonic acid can occur for example by treatment with trimethylsilyl bromide and methanol (Step 2).

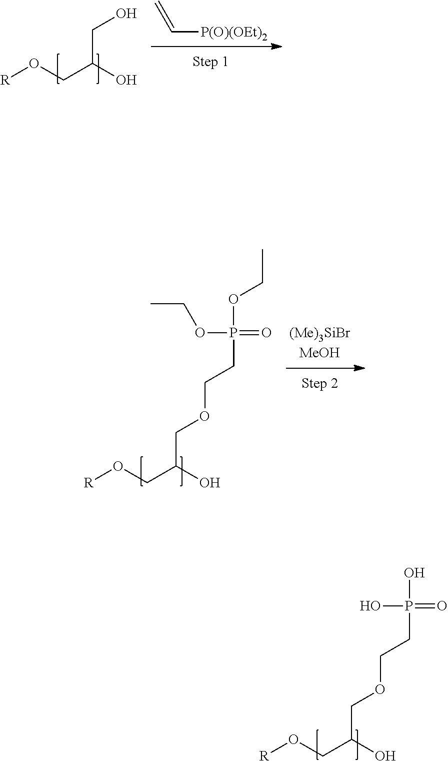

In some embodiments, the polymer backbone can be completely or partially functionalized with a substrate-coordinating group. In other words, a polymer backbone that contains alkene groups such as allyl groups (e.g., from polymerization of allyl-containing monomers) can be reacted with a supermolar or a submolar amount of substrate-coordinating group. By controlling the amount of substrate-coordinating group that is present in the reaction, the percentage of reactive functional groups that are converted to substrate-coordinating groups can be adjusted.

In some embodiments, the allyl groups of the poly(allylvalerolactone-valerolactone) copolymer can be functionalized with a bisphosphonate. For example, the scheme below shows a general process for functionalizing a poly(allylvalerolactone-valerolactone) copolymer with a bisphosphonate. In step 1, the allyl group of the poly(allylvalerolactone-valerolactone) is reacted using a thiol-ene reaction to prepare a poly(allylvalerolactone-valerolactone) conjugated to 3-mercaptopropionoic acid (MPA). In step 2, the carboxylic acid group of the 3-mercaptopropionoic acid functionality is condensed with alendronic acid (ALE) to afford a poly(allylvalerolactone-valerolactone) copolymer that is functionalized with a terminal bisphosphonate group.

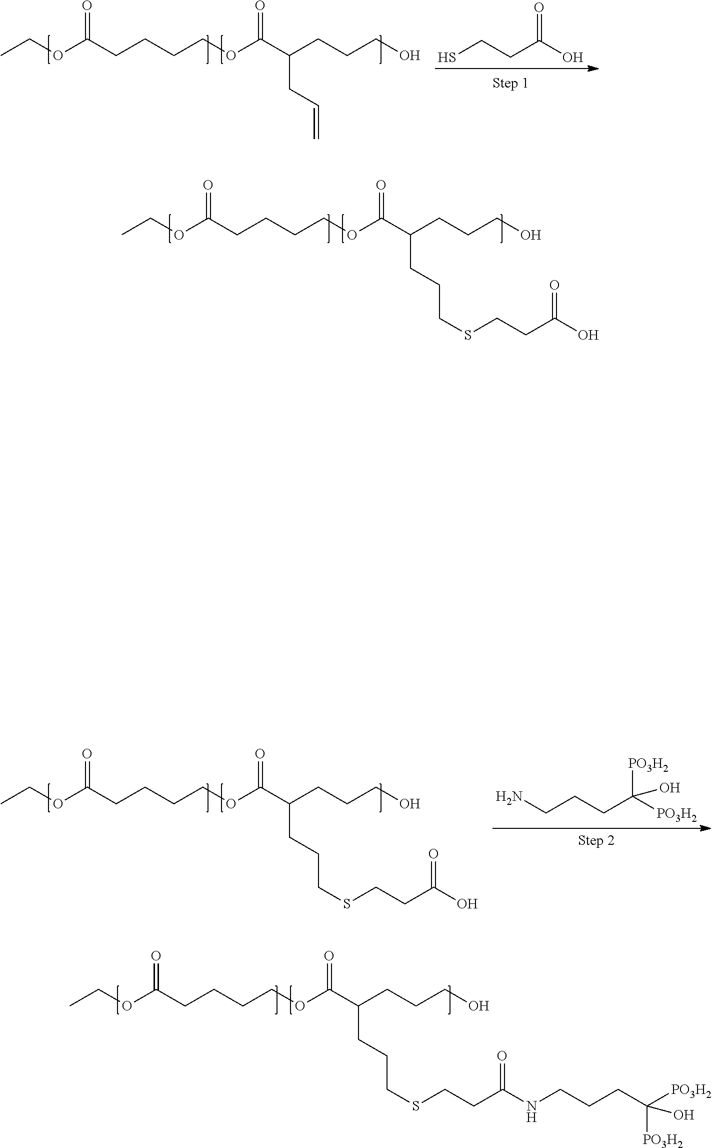

Examples 4-7, 9-11 and 13 demonstrate the functionalization of polymer backbones (e.g., polyvalerolactone or polyglycidol backbones) with alendronate substrate-coordinating groups. In some embodiments, a higher percentage of substrate-coordinating groups are used if greater substrate coverage is desired. For example, the percentage of alkene (e.g., allyl) units converted to a substrate-coordinating group such as alendronate or phosphonate can be between 1 and 100. In some embodiments, aspartic acid is used instead of alendronate. In some embodiments, NHS-DOTA is used instead of alendronate. In some embodiments, mercapto-multi-acetates or mercapto-aminopolycarboxylic acids are used instead of 3-mercaptopropionic acid.

### Reactive Functional Groups

In some embodiments, the polymer coatings of the present disclosure can be further functionalized with additional reactive functional groups. In some embodiments, the additional functional group is different from a metal-coordinating group. As depicted below, a polymer backbone such as a polyglycidol (e.g., a linear polyglycidol, a branched polyglycidol or a hyperbranched polyglycidol) can be functionalized. For example, a polymer backbone of the present disclosure can be functionalized with one or more alkenes; one or more alkynes; one or more epoxides; one or more hydroxyl groups; or combinations thereof.

Functional groups can also include norbornene and dibenzocyclooctyne acid (e.g., dibenzocyclooctyne-PEG4-acid). In some embodiments, these functional groups can be used (e.g., as functional handles) to conjugate the functional groups to additional components such as antibodies, drugs, and the like. Any of the functional groups set forth herein can be incorporated on a polymer backbone with any of the other functional groups in any combination.

In some embodiments, functional groups such as norbornene and dibenzocyclooctyne acid (e.g., dibenzocyclooctyne-PEG4-acid) can be considered conjugating groups. For example, norbornene and/or a dibenzocyclooctyne can be used as a functional handle to crosslink (e.g., with a crosslinker). In some embodiments, a conjugating group can be attached to the polymer backbone by a linker. For example, in the case of dibenzocyclooctyne-PEG4-acid, the dibenzocyclooctyne is attached to the polymer backbone using a 4-unti PEG linker. One of skill in the art will readily understand that a linker such as a PEG linker can also include other atoms and functional groups (e.g., esters) that can be necessary to covalently bond the conjugating group to the polymer backbone.

In some embodiments, the reactive functional group is a drug.

In some embodiments, the polymer backbone can incorporate the reactive functional groups by polymerizing monomers that are pre-functionalized with reactive functional groups (e.g., with allyl groups). In some embodiments, the reactive functional groups are used as shown above to conjugate the substrate-coordinating groups. For instance, allyl groups can be incorporated into a polymer backbone by using allyl-containing monomers such as allyl valerolactone. In some cases, hydroxyl groups are formed from the polymerization of glycidol.

As shown below, a polyglycidol backbone can be functionalized with an alkene group by reacting the free hydroxyl groups of the polymer backbone with, for instance, an acid chloride such as acroyl chloride or with a corresponding carboxylic acid and an ester-coupling reagent (e.g., acrylic acid and EDC/DMAP).

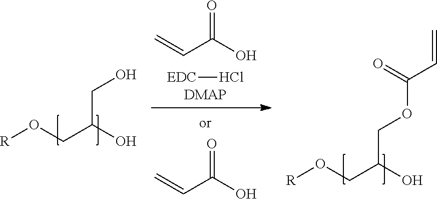

In some embodiments, a functionalized polyglycidol polymer backbone of the present disclosure can be prepared by crosslinking monomers that are pre-functionalized. For example, an allyl-functionalized polyglycidol backbone can be prepared by co-polymerizing glycidol with allyl glycidyl ether. Similarly, a functionalized polyglycidol backbone can be prepared by co-polymerizing glycidol with glycidyl acrylate.

In some embodiments, the percentage of free hydroxyl groups that are converted to alkene groups (e.g., as shown above) can be between about 1% and 100%.

### Bi-Functionalization

In some embodiments, the polymeric backbones of the present disclosure can be bifunctionalized (e.g., can be functionalized with a substrate-coordinating group and with a reactive functional group). In other words, multiple modes of functionality can be applied to the same polymeric backbone to create a di- or multi-functional polymer.

In some embodiments, the polymer backbone can be completely or partially functionalized with a functional group. For example, all of the hydroxyl groups can be converted to an alkene, or some (i.e., a fraction) of the hydroxyl groups can be converted to the alkene. This results in polymer backbones that contain both allyl (i.e., alkene) functionality as well as hydroxyl functionality. Both of these functional groups (as well as other functionality) can be incorporated into the polymer backbones in addition to the substrate-coordinating group.

As shown below, a polyglycidol backbone can be functionalized with a phosphonate via a conjugate (e.g., Michael) addition. Next, acroyl chloride can be added and reacted with the polymeric backbone to attach an ester vinyl group. Thereafter, the ethyl phosphonate can be deprotected to the corresponding phosphonic acid. Although a linear polyglycidol chain is shown below, the technique can be used for semi-branched and hyper-branched polymers. Additionally, one of skill in the art will recognize that the degree of functionality of can be adjusted (e.g., by modifying the amounts and ratios of the reagents added relative to the polymeric backbone). Accordingly, it can be possible to completely or partially functionalize the polymer backbones of the present disclosure.

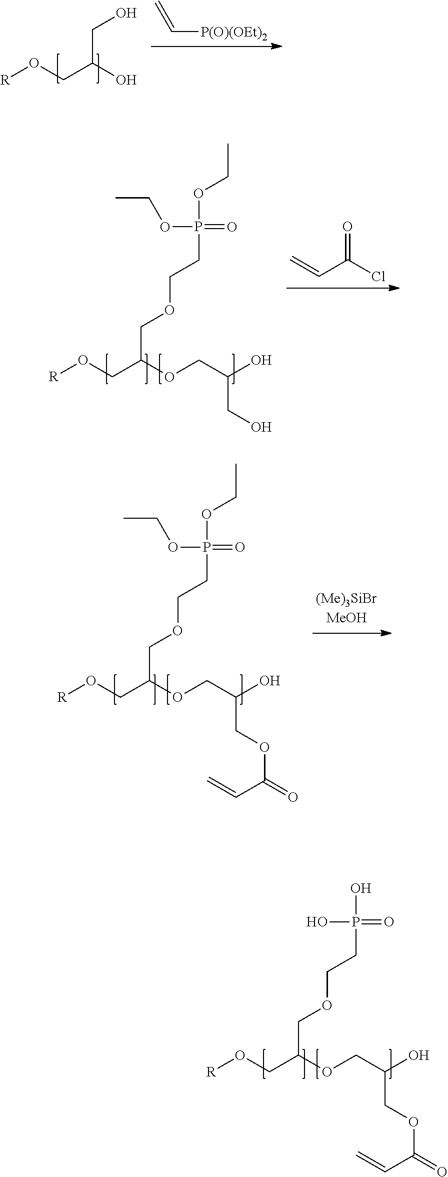

In some embodiments, a copolymer containing a functional group (e.g., an alkene functional group) can be prepared first, followed by addition of a substrate-coordinating group, as shown below.

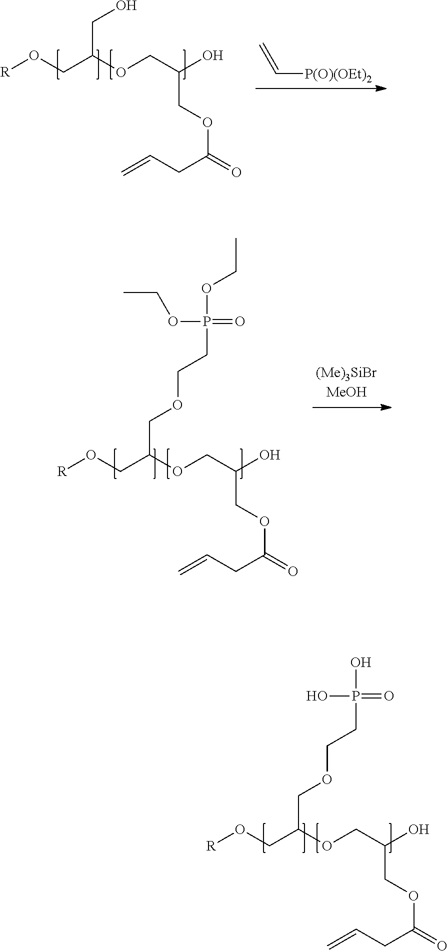

As shown in the scheme below, a bisphosphonate (e.g., alendronate) can be conjugated to a polymer backbone to form an alkene-bisphosphonate bifunctional polymer. Beginning with an alkene-functionalized homopolymer, some alkenes can be converted to carboxylic groups using a thiol-ene reaction. The alendronate can then be conjugated to the carboxylic acid using an amide coupling reaction.

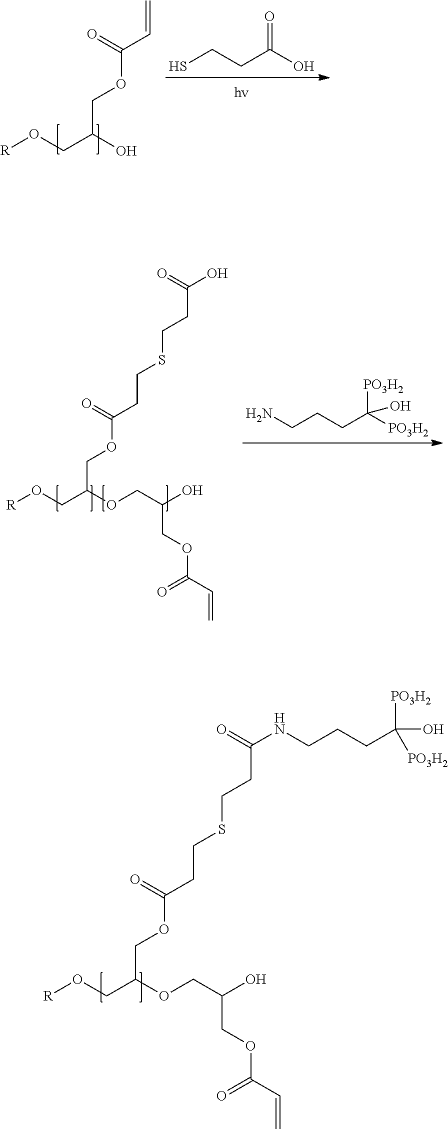

### Crosslinkers

In some embodiments, the polymeric coatings of the present disclosure can be crosslinked. For example, the polymeric backbones of the present disclosure can be crosslinked using PEG. In other words, PEG can be incorporated into the polymeric backbone of the polymeric coatings. Additionally or alternatively, PEG can also be incorporated into the polymeric coatings as a crosslinking agent.

In some embodiments, the PEG is a di-functional PEG. In some embodiments, the PEG is a multifunctional PEG. For example, the PEG can be a branched PEG. As used herein, branched PEGs can have three to ten PEG chains emanating from a central core group. In some embodiments, the PEG crosslinker can be a star PEG. As used herein, star PEGs can have 10 to 100 PEG chains emanating from a single core group. In some embodiments, the PEG crosslinker can be a comb PEG. As used herein comb PEGs can have multiple PEG chains normally grafted onto a polymer backbone (e.g., a poly(avl-vl) backbone.

In some embodiments, the PEG crosslinker can be between about, for instance, about 300 Da to about 30 kDa. In one or more embodiments, the PEG crosslinker can be branched (e.g., can be a three-arm PEG; a 4-arm PEG; or a multi-arm PEG). In some embodiments, the crosslinkers can be reversible crosslinkers. In some embodiments, the crosslinkers can be cleavable crosslinkers. For example, the crosslinkers of the present disclosure can include cleavable groups such as esters.

In some embodiments, the crosslinkers can be thiol-reactive crosslinkers such as alkenes. For example, the coatings of the present disclosure can be crosslinked with dithiols. In some embodiments, the coatings of the disclosure can be crosslinked with dithiols after the substrate surface has been coated. For instance, a thiol-modified PEG can participate in a thiol-ene reaction. In one example, an alkene-functionalized polymer of the disclosure (e.g., a polyester or a polyglycidol) can be dissolved in appropriate solvent such as water, DMSO or DMF. A radical can be added (e.g., DMPA for organic solvents or VA-044 for aqueous solvents, about 0.1 to 0.3 equivalents based on moles alkene). Next, a crosslinker can be added and the system can be irradiated with a UV light source (e.g., at 365 nm). In some embodiments, thermal initiators can also be used, wherein the reaction can be heated to initiate the reaction.

Specifically, in some embodiments, crosslinking can take place using a radial-initiated mechanism (e.g., a thiol-ene reaction). For example, free allyl groups (e.g., allyl groups that are not reacted to form a substrate-coordinating group) can be used in a crosslinking reaction. In some embodiments, a dithiol can be used as a crosslinker to crosslink the allyl groups from different polymeric backbones. For example, a di-thio PEG polymer can be used as a crosslinker. Other thiol-containing molecules and/or polymers can also be used as crosslinkers in a thiol-ene reaction (e.g., using an alkene functional group bound to the polymer backbone). For example, thiol-functionalized polyelectrolytes such as thiol-modified hyaluronic acid can be used. In some embodiments, thiol-modified gelatin can be used as a crosslinker.

In some embodiments, triallyl isocyanurate (TAIC), trimethally isocyanurate (TMAIC), trimethylolpropane triacrylate (TMPTA), 2,2-Dimethoxy-2-phenylacetophenone (DMPA), and other photoinitiators can be used to initiate the crosslinking reaction. In some embodiments, the crosslinking reaction can occur upon the addition of high energy light (e.g., UV light, blue laser light). For example, the crosslinking reaction can occur via the mechanism given below. In the scheme below, “L” is any linker (e.g., a PEG linker such as HS-PEG-SH).

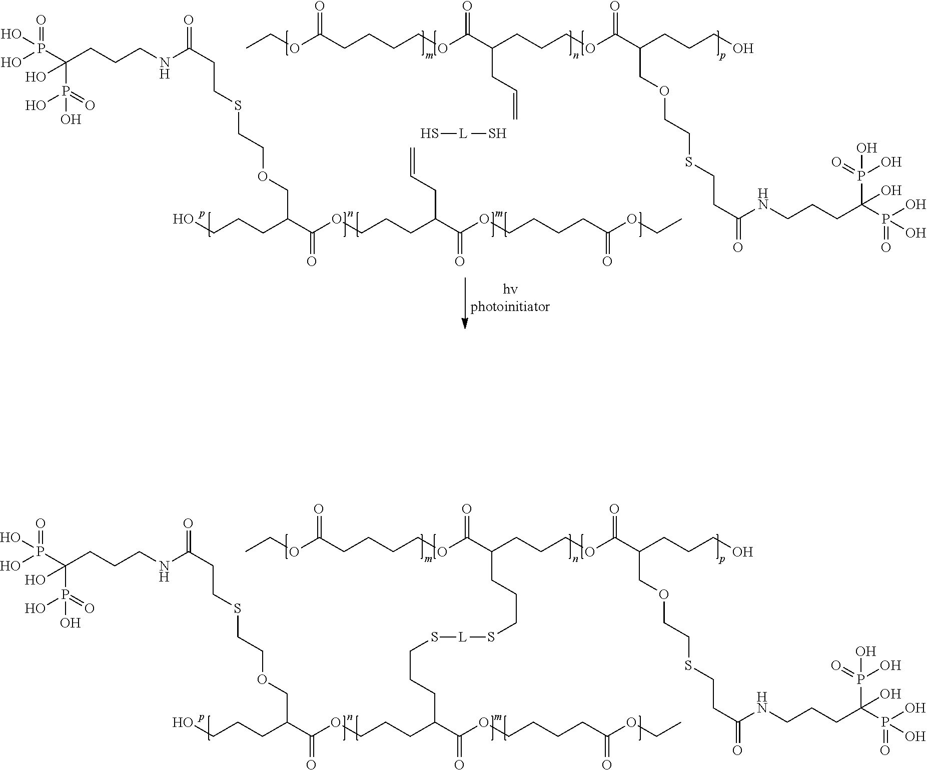

In some embodiments, other crosslinkers besides PEG can be used. For example, DNA and modified DNA, including phosphorothioated and nucleophile-terminated DNA sequences can be crosslinkers can be used.

In some embodiments, polypeptides can be used as crosslinkers. These can be derived from cellular or cell-free expression systems or from synthetic amino acid production. Polypeptides can be further modified with enzymes such as tyrosine hydroxylase to produce amino acid derivatives such as dopamine.

In some embodiments, polyesters or other biodegradable polymers as crosslinkers, this may be used. These include nucleophile-terminated valerolactone, caprolactone, lactide, glycolide, and the like. In some embodiments, other polymers besides polyesters can be used.

In some embodiments, a crosslinker can be positioned as a pendent group off of a polymer backbone (e.g., a poly(avl-vl) backbone). In some embodiments, pyridinyl groups including multivalent molecules such as a bipyridynyl or terpyridinyl can be used as crosslinkers.

In some embodiments, the crosslinking agent can induce crosslinking upon radiation exposure (e.g. UV, blue light, electron beam, or gamma radiation). In some embodiments, the crosslinking can proceed via a thiol-ene click reaction.

### Characterization of the Disclosed Polymer Coatings

The polymer coatings of the present disclosure can be used to coat a substrate as set forth herein. In some embodiments, the polymeric coatings can act as an interface between the substrate (e.g., an implant) and the surrounding environment (e.g., tissue). Accordingly, in some embodiments, the coatings of the present disclosure are hydrophilic. In some embodiments, the coatings are hydrophobic. In some embodiments, the polymer backbone of the coatings is hydrophobic, and the polymer backbone is functionalized with hydrophilic functional groups to make the coating more hydrophilic.

In some embodiments, the polymeric coatings of the present disclosure can be between about 0.001 μm and about 100 μm thick. For example, the polymeric coatings of the present disclosure can be between about 0.001 μm and about 1 μm thick. The coating thickness can be determined by visual inspection or by using an appropriate technique such as scanning electron microscopy.

In some embodiments, the polymeric coatings of the present disclosure can cover substantially all (i.e., about 100%) of the surface of a substrate. In some embodiments, the polymeric coatings can cover a portion of the surface of the substrate. For example, in some embodiments, the polymeric coatings can cover about 95%, about 90%, about 80%, about 70%, about 60%, about 50%, about 40%, about 30%, about 20%, or about 10% of the surface of the substrate.

In some embodiments, the crosslinking density can influence toughness, hardness, and/or porosity of the coating. That is, in some embodiments, greater crosslinking can lead to a coating that is harder, tougher, and/or less porous than a coating that has less crosslinking.

In some embodiments, pores (e.g., micropores) can be created by selectively crosslinking the polymer backbones of the present disclosure (e.g., using a photoinitiator and high-energy light such as UV or blue light laser) and removing (e.g., washing or rinsing away) uncrosslinked polymers. In some embodiments, the use of a photoinitiator and high-energy light can be used to crosslink polymer backbones with alkene (e.g., allyl) functionality with, for instance, dithiol crosslinkers.

In some embodiments, PEG can be incorporated into the polymer backbones of the present disclosure to increase the porosity of the coatings. For example, a copolymer such as poly(avl-vl)-co-PEG, or poly(avl-vl)-co-poly(vl-avl)[PEG])-PEG can be used. In some embodiments, the polymer backbone can have PEG groups conjugated to the pendant reactive groups (e.g., allyl groups) before the polymer backbone is deposited and crosslinked at the terminal layer. In some embodiments, the pendant PEG groups can increase the overall PEG density and decrease protein fouling or bacterial adhesion to the substrate surface.

In some embodiments a porogen can be used to increase the porosity of the polymeric coatings of the present disclosure. Porogens can be used as an additional method of controlling pore size and/or crosslinking density in a polymer (e.g., the polymeric coatings of the present disclosure). For instance, a porogen may be a crystal or material that can be incorporated into a polymeric backbone (e.g., during polymerization of the backbone) that can be subsequently removed by dissolution in a specific solvent (e.g., a solvent that does not dissolve the polymer). For instance, a salt crystal such as NaCl can be used as a porogen to block formation of a polymer in a certain three-dimensional space during polymerization of a polymer backbone as set forth herein. Next, the polymer backbone can be exposed to water to dissolve the porogen salt crystal. Similarly, a material such as paraffin can be used as a porogen and dissolved using an organic solvent.

Use of porogens can increase pore size of the polymeric coatings of the present disclosure. For example, porogens can be used to create pores (e.g., voids) in a polymeric coating of a substrate. Without wishing to be bound by theory, increases in pore size and/or increases in pore number can enable control over the properties of the polymeric coatings. For instance, pore size and/or frequency can influence the rate of degradation and/or the rate of drug delivery. For instance, without wishing to be bound by theory, increases pore number and/or pore size can enable greater solvent (e.g., water) penetration which can accelerate the degradation of the polymer coatings. In some embodiments, the porogen can be solvating or non-solvating.

The polymer coatings set forth herein can come in a variety of sizes and weights. For example, the polymer coatings can have a molecular weight of between about 1 to 100 kDa (e.g., about 10 kDa, about 20 kDa, about 30 kDa, about 40 kDa, about 50 kDa, about 60 kDa, about 70 kDa, about 80 kDa, about 90 kDa, or about 100 kDa). In some embodiments, the polymer backbone (e.g., the poly(avl-vl) backbone) can be between about 2 kDa and about 20 kDa. In some embodiments, the molecular weight of the polymer coatings is between about 1 and about 20 kDa (e.g., between about 5 and 15 kDa).

In some embodiments, the polymer coatings of the present disclosure can be self-plasticizing. In other words, in some embodiments, the coatings presented herein do not require a plasticizer.

Various techniques can be used to characterize the polymeric coatings of the disclosure. For example, X-ray photoelectron spectroscopy (XPS) and/or ellipsometry can be used. In some embodiments, XPS and/or ellipsometry sampling can be used on multiple locations on the surface of a coated substrate to evaluate the uniformity of the coating.

### Coating Durability

The physical properties of the polymer coatings of the disclosure can be assessed using a variety of techniques including those that simulate the clinical application of interest. For example, if the coating is used for an implant such as an orthopedic intramedullary device, a simulated surgical procedure using artificial or cadaveric bone can be used. The coated implant can be inserted into the intramedullary canal using methods consistent with the standard of care. The implant can then be removed (e.g., after a specified period of time), and the coating can be visually inspected and/or analyzed by a technique such as SEM to assess the extent of coating delamination from the implant.

In another method, the coated substrate can be subjected to handling in the presence of blood or serum to determine if the coating is durable enough to remain intact through a typical surgical procedure in the presence of a bodily fluid such as blood. In one example, the substrate surface can be placed in contact with a wheel covered with latex to simulate a surgical glove. The latex wheel can be attached to a motor that spins the latex wheel against the substrate surface. Serum can be applied to the latex surface and then pressure can be exerted by the spinning wheel onto the implant surface by a counter weight. The implant surface can be visually inspected for signs of delamination.

### Chemical Integrity of the Coating and Coating Components

The chemical integrity of coating components can be necessary to confirm that the manufacturing methods such as sterilization do not degrade the chemicals or provoke chemical reactions between coating constituents. The chemical integrity of the polymers, and other agents such as drug substances if applicable, can be determined by recovering the coating from the implant using an appropriate solvent. The chemical integrity of each coating component can be determined using an appropriate analytical method. For instance, Polymer molecular weight can be determined by methods such as GPC or viscosity, and polymer chemical integrity can be assessed by, e.g., NMR. Impregnated drug substances can be analyzed by a method such as HPLC/MS/MS.

### Coating Mass

In some embodiments, use of the coatings of the disclosure produce a reproducible coating level that is related to the surface area of the substrate (e.g., a medical implant). The mass of the coating relative to surface area can be determined by a number of techniques including gravimetric analysis. For example, the implant can be weighed prior to coating using an analytical balance. The weight of the implant before and after coating can be used to calculate the total mass of the coating. The mass of the coating can be divided by the total surface area of the implant to determine the mass per unit area.

In other embodiments, the coating mass is at a level that is too low for gravimetrical analysis. In these cases, the coating can be removed from the implant surface (e.g., using an appropriate solvent). The concentration of the coating components in the solvent can then be determined by an appropriately sensitive analytical method such as spectroscopy or HPLC/MS/MS. The coating mass per unit area can be determined by calculating the total mass recovered from the implant divided by the total surface area of the implant.

### Coating Uniformity

Depending on the clinical indication, substrates such as medical devices can be large enough that uniformity of the coating over the entirety of the device surface area is necessary. In one example, the thickness of the coating is determined by visual inspection using an appropriate technique such as scanning electron microscopy. Separate visual inspections can be conducted at a variety of points on the implant surface to determine the extent of variability.

In another example, the coating uniformity can be determined gravimetrically. In this method the implant can be divided into segments and the coating can be removed from each segment using a solvent that can dissolve the coating. The polymer can be recovered from the solvent through evaporation and the mass of the polymer can be determined. The mass per unit area can be determined by dividing the mass of polymer obtained from a given segment by the surface area of that segment.

If the coating contains a drug(s) or other agent, then the mass of each drug can be determined by an appropriate analytical method such as HPLC. In some embodiments, the mass of the drug recovered from a specific substrate segment can be divided by the surface area of that segment to determine the mass of the drug per unit area on the substrate.

In addition to chemical analysis, the biological activity of an impregnated drug substance can be characterized. For example, if an antibiotic is incorporated in the coating, then the minimal inhibitory concentration (MIC) for the drug against a specific pathogen can be used to confirm the specific activity of a given antibiotic. Other drugs can have definitive assays to determine biological activity. In either instance, the biological activity per unit mass can be determined to demonstrate that coating methods, sterilization and storage conditions do not alter drug activity.

### Methods of Coating Substrates

The polymeric coatings of the present disclosure can be applied to a substrate in a variety of different ways. In some embodiments, the coatings can be applied to a substrate by dip coating or spraying (e.g., spray-coating), flow coating, or using a brush or sponge. In some embodiments, a polymer comprising a substrate-coordinating group can be incubated with the substrate (e.g., a metal implant).

In some embodiments, the coatings of the present disclosure are first dissolved in a solvent prior to application to a substrate. For example, the substrate can be dipped in a solution comprising the coating, or sprayed with a solution comprising the coating. The solvent can be any solvent capable of dissolving the polymeric coatings, for instance, N-methyl-2-pyrrolidone, ethyl acetate, methylene chloride, THF, or DMF. In some embodiments, the coatings of the present disclosure are hydrophilic and the solvent is water. In some embodiments, the solvent comprises a combination of various solvents (e.g., the solvent can be a mixture of water and an alcohol such as methanol or ethanol). In some embodiments, a substrate (e.g., a metal implant) can be dipped in coatings or sprayed with coatings that are substantially free of a solvent.

In some embodiments, a substrate-coordinating group can be pre-incubated with the substrate prior to polymerization of the polymer backbone. For example, a substrate-coordinating group such as a bisphosphonate (e.g., alendronate) can be incubated with the substrate (e.g., a metal implant) to coat the surface of the substrate. Next, any unbound substrate-coordinating group (e.g., alendronate) can be rinsed away. Next, a functionalized polymer backbone (e.g., a polymer backbone that is functionalized with a carboxylic acid (—COOH) group) can be reacted with the bound substrate-coordinating group. For example, if dopamine or alendronate is used as the substrate-coordinating group, the carboxylic acid functionality of the polymer backbone can be condensed with the free amine group of the alendronate or dopamine. In some embodiments, an amide-coupling reagent such as DCC can be used to facilitate the reaction.

In some embodiments, the method of first incubating a substrate-coordinating group with a substrate prior to contacting (e.g., reacting or bonding) the substrate-coordinating group with the polymer backbone can result in multi-dentate polymers adhered to the substrate (e.g., implant) surface. In some embodiments, not all of the free functional groups (e.g., —COOH groups) can be conjugated to the bound substrate-coordinating groups. In some embodiments, the unreacted functional groups (e.g., —COOH groups) can serve as a functional handle for further conjugations (e.g., to grow the coating thickness).

In some embodiments, the substrate surface (e.g., a metal surface such as a metal implant) can be first oxidized prior to coordinating with a substrate-coordinating group of the present disclosure. For example, in some embodiments a metal surface can be oxidized and optionally silanized (e.g., to install polymerization initiation sites) For example, a polymerization initiation site can comprise a substrate-coordinating group with a free nucleophilic group such as a free amine or free hydroxyl group.

In some embodiments, a bisphosphonate such as neridronate can be used. For example, the free amine of neridronate can optionally be converted to a hydroxyl group. Next, the neridronate or hydroxyl neridronate can be bound to a substrate (e.g., via the bisphosphonate group). Next, monomers such as valerolactone and/or allylvalerolactone can be introduced and the hydroxyl group of hydroxyl neridronate or the amino group of neridronate can be used to initiate a polymerization reaction. In some embodiments, this can result in pendant-functionalized polymers. After polymerization, the resulting coating can optionally be crosslinked.

When a solvent is used to dissolve the polymer coatings of the present disclosure, the concentration of the polymer solution can be adjusted to achieve a desired viscosity. In some embodiments, a more concentrated solution of polymer can be more viscous than a less concentrated solution. Additionally, in some embodiments, the resulting thickness of the polymer coating on a substrate surface (e.g., an implant) can be controlled by adjusting the concentration (and/or viscosity) of the polymer solution. In some embodiments, using a more viscous (e.g., more concentrated) polymer solution can result in a thicker polymer film on the surface of the substrate, whereas using a less viscous (e.g., less concentrated) polymer solution can result in a thinner polymer film on the surface of the substrate. In some embodiments, the polymers of the present disclosure are dissolved at a concentration of between about 0.5 to about 2% by weight (e.g., about 0.5%; about 0.6%, about 0.7%; about 0.8%; about 0.9%; about 1.0%; about 1.1%; about 1.2%; about 1.3%; about 1.4%; about 1.5%; about 1.6%; about 1.7%; about 1.8%; about 1.9%; or about 2.0%).

Without wishing to be bound by theory, the percent of a substrate that is covered by the coatings can also be a function of the concentration of the solution used to apply the coatings. In other words, when the coatings of the disclosure are dissolved in a solvent prior to applying the coatings to a substrate, a higher concentration of coating in the solution can lead to greater coverage of the substrate. Accordingly, in some embodiments, the coatings of the disclosure can be dissolved in a solvent at high concentrations and applied to a substrate to ensure high coverage of the substrate. In some embodiments, the coatings of the disclosure can be dissolved in a solvent at low concentrations and applied to a substrate to ensure low coverage of the substrate. In some embodiments, the polymer coatings of the present disclosure can be substantially saturated in a solvent prior to exposing the substrate to a solution of polymer coating.

When a solvent is used to dissolve the coatings of the present disclosure, the solvent can be removed after application to the substrate (e.g., by dipping or spraying) by letting the solvent evaporate. In some embodiments, evaporation of the solvent can be aided (e.g., accelerated) by heating the substrate or blowing air over the substrate or using a vacuum to remove the solvent. For example, a substrate can be dipped in a solution comprising a polymeric coating and the substrate can be allowed to dry on a drying rack. In some embodiments, the solvent can be removed by wiping the substrate with a cloth or towel. For example, after a dip-coating process, the excess coating solution can be allowed to drip off the substrate and back into the reservoir. The remaining film on the substrate then consists of the remaining solvent, polymer and any other substance dissolved or suspended in the coating solution. If the solvent is volatile, it can evaporate when the substrate is removed from the coating solution, thus depositing the coating on the implant surface.

Without wishing to be bound by theory, the percent of a substrate surface that is covered by the coatings, and the thickness of the coatings, can be a function of a number of factors such as the number of substrate-coordinating groups attached to the polymeric backbone. In other words, one of skill in the art will recognize that it is possible to control the degree of coverage of the substrate by attaching more or fewer substrate-coordinating groups. Accordingly, for applications in which relatively little coverage of the substrate surface is desired, fewer substrate-coordinating groups can be incorporated into the backbone of the coatings. Alternatively, if significant coverage (e.g., substantially full coverage) of the substrate is desired, the coatings can be prepared including a higher percentage of substrate-coordinating groups.

In some embodiments, the polymer coatings described herein can be increased in thickness by polymerizing successive layers of coating. For example, multiple layers of the coatings of the disclosure can be applied to a substrate surface by performing successive crosslinking reactions. In some embodiments, the functional groups of a polymer backbone (e.g., electrophilic groups) can be used to carry out additional polymerization reactions (e.g., using free nucleophile groups). Alternatively, the functional groups of a polymer backbone (e.g., nucleophilic groups) can be used to carry out additional polymerization reactions (e.g., using free electrophilic groups).

In some embodiments, a design of experiment can be used to arrive at the desired parameters such as concentration of the coating solution; number of substrate-coordinating groups on the polymer backbone; amount of coating solution used to coat the substrate; and the amount of exposure time of the substrate to the coating solutions. These parameters can be adjusted to control the properties of the resulting polymer coatings.

Without wising to be bound by theory, the percent coverage of a substrate can also be a function of the time that the substrate is exposed to the coatings or solution of the coatings. For example, in some embodiments, spraying a substrate with a solution of the coatings for a longer period of time can lead to greater coverage of the substrate, whereas spraying the substrate with a solution of the coatings for a shorter period of time can lead to lesser coverage of the substrate.

For example, a polymeric coating of the disclosure (e.g., ALE-PVL-AVL) can be dissolved in a solvent (e.g., DMSO, for example at about 2.5% wt/wt) to form a solution. Next, a substrate such as a metal coating can be placed in the solution and optionally agitated (e.g., for about an hour).

In some embodiments, the percent of a substrate surface that is covered by the coatings, and the thickness of the coatings, can be a function of the amount of coating solution applied to the substrate (e.g., by dip-coating or spray coating). For example, applying more of a polymer solution to a substrate can result in greater (e.g., thicker and more dense) coverage of the substrate.

In some embodiments, the coatings of the present disclosure self-adhere to the substrate after application to the substrate. For example, in some embodiments the substrate-coordinating groups contained on the polymer backbone of the coatings can automatically interact with and coordinate to a substrate. Accordingly, in some embodiments, it is unnecessary to cure the coatings of the present disclosure to the substrate.

In some embodiments, an additional component can be incorporated into a solution comprising a polymer coating of the present disclosure. For instance, a drug or other therapeutic agent can be incorporated into the solution, and thus can be incorporated onto the substrate coating after application of the solution.

In some embodiments, the drug and/or agent is soluble in the solvent (e.g., ethyl acetate) and the coating solution is a uniform solution consisting of the polymer and the drug and/or other agent.

In another embodiment, the drug or other agent is insoluble in the solvent (and thus the coating solution) and the resulting mixture is a suspension. In this instance, the suspension can be agitated (e.g., by stirrers or mixing pumps) so that the insoluble substances do not settle out of the coating solution.

The particle size of the drug or other agent can be adjusted by physical milling or air jet milling so that the particle size is appropriate for the specific coating application. In some embodiments, the drug or other agent in the coating solution can be refrigerated to maintain stability. To achieve temperature control, the reservoir for the coating solution can be refrigerated using a thermostat controlled refrigeration system. The coating solution can be analyzed for physical stability with respect to polymer chemical stability and the stability of any substance incorporated in the coating solution. The stability analysis can determine the shelf life of the coating solution to ensure that the resulting polymer coatings have the expected physical and chemical properties.

### Dip Coating Process

In some embodiments, a coating solution (e.g., a solution of the polymer in ethyl acetate) is placed in a reservoir for the dip coating process. Dip coating can be initiated by fixing the substrate (e.g., a medical device or implant) to be coated onto a fixture such that the substrate can be submerged into the coating solution in a controlled and reproducible manner. The fixture can be integrated with a stepper motor coupled to a motor controller that moves the implant in and out of the coating solution at a controlled immersion and extraction rate. In some embodiments, a slower immersion and extraction rate can result in a thicker polymer film. In contrast, in some embodiments, a faster immersion and extraction rate can result in a thinner polymer film

### Spray Coating Process

Ultrasonic spray coating can be applicable for applying coatings to substrates such as medical devices (e.g., those with complex surface geometries). Polymers with or without a drug or other agent can be dissolved in an appropriate solvent (e.g., ethyl acetate). In some embodiments, the coating solution is loaded into syringes which are controlled by syringe pumps that use precisely controlled stepper motors. The syringe pumps can deliver the coating solution to an ultrasonic spray nozzle that can mix the coating solution with gas and the resulting mixture can be converted to small droplets by ultrasonic energy. The polymer droplets can be directed at the implant surface and adhere to the surface with the concomitant evaporation of the solvent. When the solvent evaporates, the polymers along with any added drugs or agent can form a coating. In some embodiments, the implant that is spray coated is held by a fixture which rotates, and the spray nozzle can also articulate to ensure an even coating.

In some embodiments, the coating thickness can be adjusted by controlling the viscosity (i.e., the concentration) of the coating solution, the rate of the syringe pump stepper motor, the geometry of the spray nozzle, the rate of gas inflow and/or the power level of the ultrasonic energy.

### Methods of Using the Disclosed Compounds

The coatings of the present disclosure can be used for a variety of applications. In some embodiments, the coatings can be used to cover a substrate such as a medical device (e.g., a medical implant). The coatings can be used to impart chemical and/or physical properties to the substrate to which they are adhered.

In some embodiments, the coatings of the present disclosure can be bifunctional or multi-functional (e.g. trifunctional, tetra-functional). In other words, the coatings can have a substrate-coordinating functionality (e.g., a metal-coordinating group such as a bisphosphonate or catechol) along with additional functionality (e.g., an alkene functional group). In some embodiments, the additional functionality can be used to impart the desired properties of the coatings.

In some embodiments, the coatings of the present disclosure can have functional groups that are oriented in different directions. For example, the substrate-coordinating groups of the coatings can be oriented toward the substrate, while the additional functional group can be oriented away from the substrate (i.e., toward to surrounding environment).

In some embodiments, the coatings described herein can be degradable. For example, the polymer backbones can be degradable under a variety of mechanisms such as hydrolysis, alcoholysis (e.g., ethanolysis), or under the action of an enzyme. The coatings described herein can be degradable in vivo or ex vivo. In some embodiments, the properties of the coatings can be tailored to allow the coatings to degrade in a certain period of time (e.g., within 6 months, within 5 months, within 4 months, within 3 months, within 2 months, within 1 month, within 4 weeks, within 3 weeks, within 2 weeks, within 1 week, or within less than a week). In some embodiments, the polymer backbone can be tailored to enable degradation of the coatings. In some embodiments, the crosslinking agent (e.g., PEG) can be tailored to enable degradation of the coatings. In some embodiments, both the crosslinking agents and the polymer backbones are tailored to enable degradation of the coatings. Without wishing to be bound by theory, tailoring the coatings herein to enable degradation can include incorporating reactive groups into the coatings that can be ruptured under certain conditions. For examples, the polymer backbones and/or crosslinking groups can incorporate reactive groups such as esters that can be cleaved in the presence of water or a nucleophile. For example, incorporating ester groups into the polymer backbone or the crosslinker can provide polymeric coatings that are susceptible to slow hydrolytic breakdown, with the result that the coatings undergo slow degradation and dissolution.

In some embodiments, degradation of the polymeric coatings described herein can occur as a function of the number of degradable functional groups incorporated into the coatings. For example, incorporating a high number of ester linkages can provide a coating that degrades more rapidly (e.g., in vivo) than a coating that incorporates fewer ester linkages.

For applications in which slower degradation is desired, the polymeric coatings described herein can include fewer degradable groups. In place of degradable (e.g., biodegradable, hydrolysable) groups such as esters, other linkers such as ethers can be used. Such groups can be less susceptible to degradation via hydrolysis or enzyme cleavage and can impart greater stability of the coatings of the present disclosure.

In some embodiments, the polymeric coatings of the present disclosure can be bioabsorbable. In some embodiments, bioabsorbable polymers include polyvalerolactone (PVL), poly(allyl-valerolactone) (PAVL), poly(L-lactide) (PLLA), poly(D-lactide) (PDLA), polyglycolide (PGA), polymandelide (PM), polycaprolactone (PCL), poly(trimethylene carbonate) (PTMC), polydioxanone (PDO), poly(4-hydroxy butyrate) (PHB), and poly(butylene succinate) (PBS), poly(DL-lactide) (PDLLA), and poly(L-lactide-co-glycolide) (PLGA).

In some embodiments, the polymeric coatings of the present disclosure can include degradable functional groups. For example, degradable functional groups can be derived from monomers that include, but are not limited to valerolactone, L-lactic acid, glycolic acid, caprolactone, dioxanone, D-lactic acid, mandelic acid, trimethylene carbonate, 4-hydroxy butyrate, and butylene succinate.

In some embodiments, the porosity of the coatings can be adjusted (e.g., by varying the amount of crosslinking between the polymer backbones of the coatings). That is, as set forth above, the crosslinking of the coatings can influence the hardness and/or porosity of the coatings. In some embodiments, micropores in the coatings can improve the loading of additional functionality to the coatings (e.g., an advanced pharmaceutical intermediate such as a drug).

### Point-of-Care Coating: User Application

In some embodiments, the polymer coatings described herein can be applied to a substrate (e.g., a medical device) shortly before use of the substrate. For example, the polymer coatings can be applied by a health care professional such as a doctor. In some embodiments, such point-of-care application can allow for increased flexibility of the use of the coatings. For example, a medical professional can apply the coating to a substrate surface prior to the substrate's use in a medical procedure. The coating solution can be opened at the point-of-care and applied to the substrate surface using an applicator brush or sponge. Alternatively, the coating solution can be provided in a spray bottle which is used to spray the coating solution onto the substrate surface. As described above, the solvent can then evaporate, leaving a polymer coating on the substrate (e.g., the medical implant).

In some embodiments, user application of the present coatings can enable the user to tailor the location of the applied coatings depending on the use of the coatings. For example, when the coating is applied to the implant surface at the point-of-care, the user can specifically apply the coating to areas of the implant that are particularly prone to bacterial colonization instead of coating the entire implant. For example, the user may apply the coating to the exposed surfaces of an orthopedic implant and not to areas of the implant that will be in direct contact with bone so as not to interfere with osseous ingrowth onto the implant.

### Point-of-Care Coating: Structured, Oriented Polymer Films

Polymer films can be fabricated to be applied to an implant surface at the point-of-care. In some embodiments, the user (e.g., a medical professional) is provided with a polymer film and directed to cover the substrate surface with the film to create a coated substrate (e.g., a coated implant). The polymer film can be engineered to have a substrate-adherent side and an outer layer that is exposed to the environment surrounding the substrate (e.g., the implant). In some embodiments, the substrate surface binding layer can be designed to adhere to a metal substrate through the incorporation of metal binding pendant groups such as bis-phosphonate moieties (e.g., alendronate).

For example, a user such as a medical professional can open a package containing dry polymer coating and hydrate the polymer film using sterile water or saline. The polymer film can be placed over the substrate surface and can adhere to the surface because of the metal-coordinating chemical moieties. In some embodiments, the user can differentiate the implant-contacting surface of a substrate from the body-contacting surface of the substrate by a color difference.

The polymer surface exposed to the body can be designed to impart a therapeutic effect. In one embodiment, the outer surface of the polymer can have a lubricious and hydrophilic layer comprising a hydrophilic polymer such as polyethylene glycol. This hydrophilic outer surface can decrease bacterial colonization to decrease the risk of implant-related infections.

In another example, the lubricious surface can decrease the force of friction between the implant surface and the body which can be useful in the case of, for instance, a catheter.

Antimicrobial Coatings

In some embodiments, the coatings of the present disclosure can have antimicrobial properties. In some embodiments, the coatings described herein can be naturally antimicrobial. That is, in some embodiments the coatings described herein can have antimicrobial properties even if the functional groups are not used to bind an antimicrobial agent. For example, without wishing to be bound by theory, the coatings of the disclosure can create a layer of water in the environment surrounding the substrate surface that can prevent bacteria from binding.

In some embodiments, the coatings of the present disclosure can be functionalized with an antimicrobial agent. In one embodiment, the coating solution can contain an antibiotic to decrease the risk of implant-related infections. For example, an antimicrobial agent can be conjugated to the coatings described herein by binding the antimicrobial agent to a functional group (e.g., an alkene) attached to the polymer backbone. As used herein, a microbe is any microorganism (e.g., a microorganism capable of infecting a host). For example, microorganisms can be bacteria, fungi, viruses, and the like.

In some embodiments, an antimicrobial agent can be loaded into a polymer coating (e.g., in an amorphous or crystalline state). Exemplary antimicrobial agents include but are not limited to gentamicin, penicillin, rifampicin, azithromycin, bleomycin, vancomycin, tetracyclines, methicillin, b-lactamase inhibitors, carbapenems, cephalasporins, and combinations thereof.

In some embodiments, the coatings can be functionalized with an antimicrobial agent that can be released (e.g., in vivo). In some embodiments, the antimicrobial agent can be an antibiotic that can be released, for instance, from an implant coating to mitigate infections.

Thus, in some embodiments, the coatings of the present disclosure can be used to prevent infections. For example, the coatings of the present disclosure can be used to coat a medical implant (e.g., a catheter) and the antimicrobial properties of the coatings can help reduce the risk of a pathogenic microbe interacting the implant and infecting the host. In some embodiments, the coatings of the present disclosure can help prevent microbes from spreading (e.g., the coatings can be bacteriostatic). In some embodiments, the coatings of the present disclosure can help kill the microorganisms on the substrate (e.g., can be bactericidal). In some embodiments, the coatings of the present disclosure can inhibit biofilm production and/or biofouling. In some embodiments, the coatings of the present disclosure can reduce infection (e.g., due to implanting a contaminated implant).

### Drug Delivery

In some embodiments, the coatings of the present disclosure can be used to deliver drugs or other therapeutic agents when bound to the substrate. For example, in some embodiments, the coatings of the disclosure can be adhered to the substrate by the substrate-coordinating group. In other words, the coatings of the present disclosure can be used as part of a polymer-drug conjugate. In some embodiments, the functional group of the coatings can then be used to bind a drug or therapeutic agent that can be released from the coatings. In some embodiments, the drug or therapeutic agent can be released in vivo. For example, the drug can include small molecule drugs such as antibiotics or macromolecules an antibodies. In some embodiments, the coatings of the disclosure can release osteoinductive drugs, anti-inflammatory drugs; TGF-b agonists; nucleosides; nucleotides; chemically-modified nucleotides; other immune-modulating drugs; and combinations thereof.

In some embodiments, a drug can be attached to the polymer backbone by a linker. For example, a PEG group can be used to attach a drug to a polymer backbone. For instance, a drug can be attached to a polymer backbone using a PEG linker much like a conjugating group such as dibenzocyclooctyne can be attached to a polymer backbone using a PEG linker. One of skill in the art will readily understand that a linker such as a PEG linker can also include other atoms and functional groups (e.g., esters) that can be necessary to covalently bond the conjugating group to the polymer backbone.

In some embodiments, the drug can be, for instance, a nucleic acid; a steroid, or an antineoplastic agent. For example, the outer layer of a polymer coating of the disclosure can contain a drug such as an antibiotic that can be released from the implant surface after implantation in the body to decrease the risk of implant-related infections. Exemplary antibiotics that can be used with the polymer coatings described herein include but are not limited to vancomycin, cefazolin, amoxicillin; doxycycline; cephalexin; ciprofloxacin; clindamycin; metronidazole; azithromycin; sulfamethoxazole/trimethoprim; amoxicillin/clavulanate; and lev ofloxacin.

### Bone Regeneration

In some embodiments, the polymeric coatings can be used for bone regrowth and/or regeneration. For example, in some embodiments, the bisphosphonate substrate-coordinating groups of the present disclosure (e.g., alendronate) can be used to enhance bone regrowth. In some embodiments, the coatings of the present disclosure can release the bisphosphonates such as alendronate in vivo.

In some embodiments, the coatings of the present disclosure can also or additionally be used to release growth factors for bone regrowth.

Accordingly, in some embodiments, the coatings of the present disclosure can be used for implants that are adhered to bone. For example, the coatings can be used to coat structural implant segments such as plates, rods and/or screws (e.g., for trauma patients or patients). In some embodiments, the patient has undergone a surgery that includes damaging a bone, such as total knee replacement or total hip replacement. In some embodiments, the coatings of the present disclosure can be regenerative.

### Hydrogel Supplements

In some embodiments, the coatings of the present disclosure can impart beneficial properties (e.g., bone resorption properties) on other coatings such as hydrogel coatings that do not inherently have such properties. For example, a hydrogel can comprise functionalized polysaccharides and/or PEG to create a porous network. The polymeric coatings can be used to help adhere a hydrogel to the surface of a substrate. That is, in some embodiments the polymeric coatings can interact with a hydrogel (e.g., can be bound to a hydrogel through the functional group) and can have additional functionality such as a bisphosphonate group that is capable of interacting with a substrate. Thus, in some embodiments, the coatings of the present disclosure can act as a glue or binding layer between a substrate and a hydrogel.

### Polymersomes and Stable Nanoparticles

In some embodiments, the coatings of the present disclosure can be used to prepare polymersomes and stable nanoparticles. In some embodiments, the polymersomes and stable nanoparticles can comprise crosslinkable and/or amphiphilic polymers (e.g., poly(avl-vl)-PEG; PEG-poly(avl-vl)-PEG; poly(avl-vl)-PEG-poly(avl-vl)).

As used herein, a polymersome can be similar to a liposome, but can have a polymer layer in place of the lipid bilayer of a liposome. Accordingly, the polymer coatings of the present disclosure can be used to prepare vesicles. In some embodiments, the polymer coatings can be used to prepare polymersomes comprising a monolayer of the polymer coating. In some embodiments, the polymer coatings can be used to prepare polymersomes comprising a bilayer of the polymer coating.

In some embodiments, when the coatings of the present disclosure are used to prepare polymersomes, the resulting polymersomes can incorporate therapeutic agents such as nutrients and/or pharmaceutical drugs. Accordingly, as used herein, the polymersomes of the present disclosure can be used as agents for drug delivery.

In some embodiments, polymersomes are made from a PEG-PVL-AVL or acylated/diacylated PGY-AGY copolymer. Without wishing to be bound by theory, polymer coatings of the present disclosure can be amphiphilic and can self-assemble into a bilayer. In some embodiments, polymer coatings of the present disclosure enable the polymersomes to be crosslinked in the radial direction with high density.

### Primer Layer for Lubricious Coating

In some embodiments, the coatings of the present disclosure can be used to adhere a lubricious coating to the substrate. For example, in some embodiments, the coatings of the disclosure can be adhered to the substrate by the substrate-coordinating group. In some embodiments, the functional group of the coatings can then be used to bind a lubricious coating, resulting in a lubricious coating bound to the substrate. Accordingly, in some embodiments, the coatings of the present disclosure can serve as a primer layer for a lubricious coating.

For example, in some embodiments, the lubricious coating can comprise a hydrogel (e.g., a polyvinylpyrrolidone (PVP) hydrogel). For example, the PVP can be entrapped within the crosslinked polymer matrix. Similarly, polyelectrolytes (e.g., hyaluronic acid) can be entrapped within the polymer coatings of the disclosure to make the resulting coatings more lubricious. The polymer coatings of the present disclosure can be used to adhere lubricious coatings (e.g., hydrogels) to medical devices. For instance, the medical devices can be, e.g., implants or wires. In some embodiments, the medical device is a guide wire (e.g., for angioplasty). Accordingly, in some embodiments, the polymer coatings of the present disclosure are biodegradable and/or biocompatible. Additionally, in some embodiments the lubricious coatings (e.g. a hydrogel such as a PVP hydrogel) are also biodegradable and/or biocompatible.

### Protein Modification

In some embodiments, the polymeric coatings of the present disclosure can be used to modify proteins. For example, the coatings can be used to prepare polyglycidyl, or polyglycidyl-hydrogel-protein modification.

### Experimental Process for Preparing Coatings

In some embodiments, the polymeric coatings of the present disclosure can be prepared according to a process comprising steps of first synthesizing a polymer backbone and subsequently modifying the polymer backbone to functionalize the same (e.g., with a substrate-coordinating group and additional functionality). Alternatively, monomers that incorporate a substrate-coordinating group and/or a reactive functional group can be polymerized directly.

### First Exemplary Experimental Process

Set forth below is a first exemplary experimental process for preparing coatings of the disclosure. First, in some embodiments, a polymer backbone (e.g., a p(avl-vl) polymer backbone can be prepared. Next, the polymer backbone can be modified. For example, the polymer backbone can be modified to include a substrate-coordinating group such has a thiolate of a bisphosphonate (e.g., alendronate). For example, the thiol-alendronate can be conjugated to the polymer backbone (e.g., the p(avl-vl) backbone using a percentage of the allyl groups on the backbone as the reactive functionality for attachment. In some embodiments, substantially all of the functional groups (e.g., the allyl groups) can be used to attach the substrate-coordinating group such as the thiol-alendronate. In some embodiments, less than all of the functional groups are used to attach the substrate-coordinating groups. In such cases, the remaining (i.e., unreacted) functional groups such as the allyl groups are left available as reactive functional groups for further elaboration of the polymer backbone. In some embodiments, about 90%, about 80%, about 70%, about 60%, about 50%, about 40%, about 30%, about 20%, or about 10% of the functional groups are reacted with a substrate-coordinating group. In some cases, between about 20% and about 80% of functional groups (e.g., allyl groups) are converted to substrate coordinating groups. In some embodiments, between about 30% and about 70%, or between about 40% and about 60% of functional groups (e.g., allyl groups) are converted to substrate coordinating groups.

Next, in some embodiments, the polymer backbone, functionalized with a substrate-coordinating group can be attached to the surface of the substrate (e.g., a metal substrate). The attachment process can comprise incubating the functionalized polymer backbone (e.g., in the presence of a solvent) with the substrate. The attachment process can also comprise dip-coating or spray coating as set forth above.

Next, in some embodiments, the polymeric backbones can be crosslinked. For example, once the polymeric backbone has been adhered to a substrate, a crosslinking reaction can take place to crosslink the adhered polymer backbones into a polymer coating. In some embodiments, the crosslinking reaction comprises a thiol-ene reaction comprising adding additional p(avl-vl), a dithiol, and a photoinitiator, and carrying out a photoinitiated crosslinking reaction. For example, the unreacted allyl groups of the polymeric backbone can react with a the sulfur atom of dithiol under a radical mechanism to produce a crosslinked polymeric coating wherein the crosslinks are the thioether linkages resulting from the thiol-ene reaction. In some embodiments, the crosslinked, adhered coatings can be purified.

In some embodiments, above-steps can be repeated using a different substrate-coordinating group (e.g., a catechol such as dopamine) or with a different crosslinking reaction.

In some embodiments, the resulting polymeric coatings can be characterized. For example, the properties of coatings comprising different polymeric backbones can be compared. For example, coatings comprising a p(avl-vl) backbone can be compared to coatings comprising a PLGA and/or a PCL backbone. In some embodiments, characterization comprises a freeze-fracture SEM; a scratch test; and/or AFM. In some embodiments, characterization comprises measurement of the drug loading and release. In some embodiments, characterization comprises a calvarial defect model or other in vivo model.

### Second Exemplary Experimental Process

Set forth below is a second exemplary experimental process for preparing the coatings of the disclosure. In some embodiments, the polymeric backbones of the present disclosure can be polymerized in a solvent (e.g., an organic solvent such as chloroform, THF, or DMSO). In some embodiments, the concentration and/or temperature of the polymerization reaction can be adjusted to obtain a desired viscosity and coating thickness. For example, a viscosity modifier (e.g., poly (vinyl alcohol)) can be added. In some embodiments a higher viscosity and concentration can lead to a thicker or fuller coating of the substrate when the substrate is contacted with the solution comprising the polymeric backbone.

In some embodiments, the substrate (e.g., a metal implant) can be coated (e.g., dip-coated) with the polymer backbone (e.g., in cases where the substrate-coordinating groups have been incorporated into the polymer backbone either after polymerization of the backbone, or because the substrate-coordinating group was present in the monomers of the polymer backbone).

In some embodiments, the solvent can then be removed from the substrate (e.g., by drying, heat, vacuum, or a combination thereof). In some embodiments, the substrate can be re-coated after the first or subsequent coating.

In some embodiments, a second polymer can be prepared in a second solvent. In some embodiments, the second solvent can also contain a solution of crosslinker and photoinitiator. In some embodiments, the second polymer can be the same as the first polymer. In some embodiments, the second polymer can be different from the first polymer (e.g., the second polymer can be a PEG copolymer). The second solvent can be an organic solvent such as THF, DMSO, or chloroform). The crosslinker can be a dithiol. In some embodiments, the crosslinker is a nucleophile including an alpha nucleophile. In some embodiments, the photoinitiator is DMPA.

In some embodiments, the coated implant can be coated again (e.g., partially coated) with the second polymer solution. In some embodiments, the second coating procedure can be the same as or different from the first coating procedure (e.g., dip coating, spray coating, or flow coating).

In some embodiments, the two polymer backbones can then be crosslinked (e.g., by the application of high-energy light such as UV light or blue laser light). The remaining uncrosslinked polymer can then be removed (e.g., through washing or rinsing).

In some embodiments, the resulting crosslinked polymer coating can then be loaded with a further active agent such as a drug or other API (e.g., a drug or antibody).

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1—Synthesis of Poly(allylvalerolactone-valerolactone) Polymers Comprising about 10% Allyl(valerolactone)

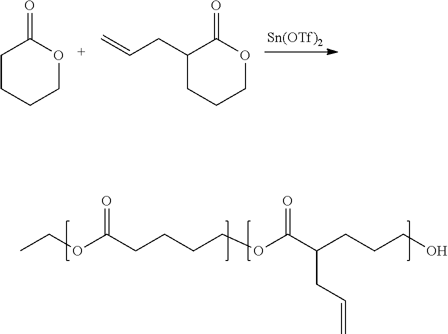

A stock solution of anhydrous ethanol in anhydrous methylene chloride was prepared (1.0 ml ethanol into 19 ml of CH₂Cl₂, 1:20 dilution) in a 25 mL flame dried and argon-purged round bottom flask. A flame dried 50 mL round bottom flask was equipped with a stir bar, sealed with rubber septum and argon purged for 10 minutes. Sn(OTf)₂ (98.6 mg, 0.237 mmol) was directly added into the flask. The reaction flask was capped with septum again and purged once more with argon for 10 minutes. Then 3.6 ml of anhydrous methylene chloride was added into the reaction flask via syringe under argon to offset the created pressure. 1.24 ml of the stock ethanol solution (1.06 mmol) was added into the reaction flask via syringe under argon.

The initiator/catalyst solution was stirred at room temperature for 30 minutes. To the stirring solution, α-allyl(valerolactone) (2.1 ml, 16.2 mmol) was added via syringe, followed by addition of 5-valerolactone (6.05 ml, 65.3 mmol) via syringe under argon balloon to offset pressure. The reaction system was stirred vigorously for 48 hours at room temperature under argon.

The resulting polymer (almost solidified) was diluted with 5 mL of methylene chloride and purified by dropwise addition into 100 ml of chilled methanol and chilled overnight in a freezer. The precipitate was rinsed with ice-cold methanol (100 ml×3) and collected by centrifuge. After overnight drying by vacuum, the final polymer product was made as a white waxy solid. Yield: 7.90 g/90.0%, (98.8% pure/NMR). NMR data analysis shows the polymer molecular weight at 14.9 KDa and avl content at 13.3% by molar. ¹H NMR (500 MHz, CDCl₃/TMS, ppm) δ: 5.72 (m, H₂C═CH—), 5.04 (m, H₂C═CH—), 4.09 (m, —CH₂—O—), 3.63 (m, CH₃CH₂O—), 2.34 (m, vl, —CH₂CH₂C(O)O—, avl, H₂C═CHCH₂CH—, H₂C═CHCH₂CH—), 1.68 (m, vl, —OC(O)CH₂CH₂CH₂—, avl, —OC(O)CHCH₂CH₂—), 1.27 (t, CH₃CH₂O—).

### Example 2—Synthesis of Poly(allylvalerolactone-valerolactone) Polymers Comprising about 20% Allyl(valerolactone)

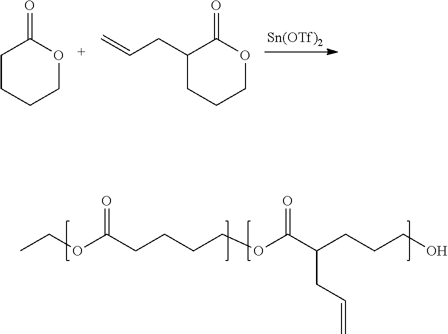

A stock solution of anhydrous ethanol in anhydrous methylene chloride was prepared (1.0 ml ethanol into 19 ml of THF, 1:20 dilution) in a 25 mL flame dried and argon purged round bottom flask. A flame dried 50 mL round bottom flask was equipped with a stir bar, sealed with rubber septum and argon purged for 10 minutes. Sn(OTf)₂ (148 mg, 0.356 mmol) was directly added into the flask. The reaction flask was capped with septum again and purged once more with argon for 10 minutes. Then 3.7 ml of anhydrous methylene chloride was added into the reaction flask via syringe under argon to offset the created pressure. 1.86 ml of the stock ethanol solution (1.59 mmol) was added into the reaction flask via syringe under argon.

The initiator/catalyst solution was stirred at room temperature for 30 minutes. To the stirring solution, α-allyl(valerolactone) (4.2 ml, 32.4 mmol) was added via syringe, followed by addition of 5-valerolactone (6.05 ml, 65.3 mmol) via syringe under argon balloon to offset pressure. The reaction system was stirred vigorously for 48 hours at room temperature under argon.

The resulting polymer (very viscous) was diluted with 5 mL of methylene chloride and purified by drop wise addition into 100 ml of chilled methanol and chilled overnight in the freezer. The precipitate was rinsed with ice-cold methanol (100 ml×3) and collected by centrifuge. After overnight drying by vacuum, the final polymer product was made as a white waxy solid. Yield: 9.80 g/88.5%, (99.3% pure/NMR). NMR data analysis shows the polymer molecular weight at 5.75 KDa and avl content at 21.7% by molar. ¹H NMR (500 MHz, CDCl₃/TMS, ppm) δ: 5.73 (m, H₂C═CH—), 5.04 (m, H₂C═CH—), 4.09 (m, —CH₂—O—), 3.64 (m, CH₃CH₂O—), 2.36 (m, vl, —CH₂CH₂C(O)O—, avl, H₂C═CHCH₂CH—, H₂C═CHCH₂CH—), 1.69 (m, vl, —OC(O)CH₂CH₂CH₂—, avl, —OC(O)CHCH₂CH₂—), 1.27 (t, CH₃CH₂O—).

### Example 3—Synthesis of Pentablock Copolymer (PAVL-b-PVL-b-PEG-b-PVL-b-PAVL)

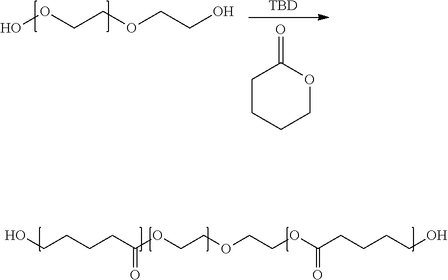

Pentablock copolymers were prepared via ring opening polymerization of VL and AVL in the presence of PEG (polyethylene glycol) as the macroinitiator and TBD (1, 5, 7-triazabicyclo[4.4.0]dec-5-ene) as the catalyst. VL (δ-valerolactone) and AVL (allyl δ-valerolactone) monomers were distilled over CaH₂ under reduced pressure and stored under argon before use. For the synthesis of PAVL-b-PVL-b-PEG 20K-b-PVL-b-PAVL, PEG 20K (1 g, 0.1 mmol of OH group) in round two-neck flask was carefully flame-dried to melt PEG and remove residue water under vacuum. After cooling to room temperature, TBD (25 mg, 0.18 mmol) was added and dried again under vacuum. The reaction mixture was dissolved in anhydrous toluene (20 mL) and stirred at room temperature for 30 min. Then, purified VL (0.5 mL, 5.0 mmol, target repeating unit=100) was transferred to the reaction mixture by cannulation to start polymerization followed by stirring at room temperature for 3 hours.

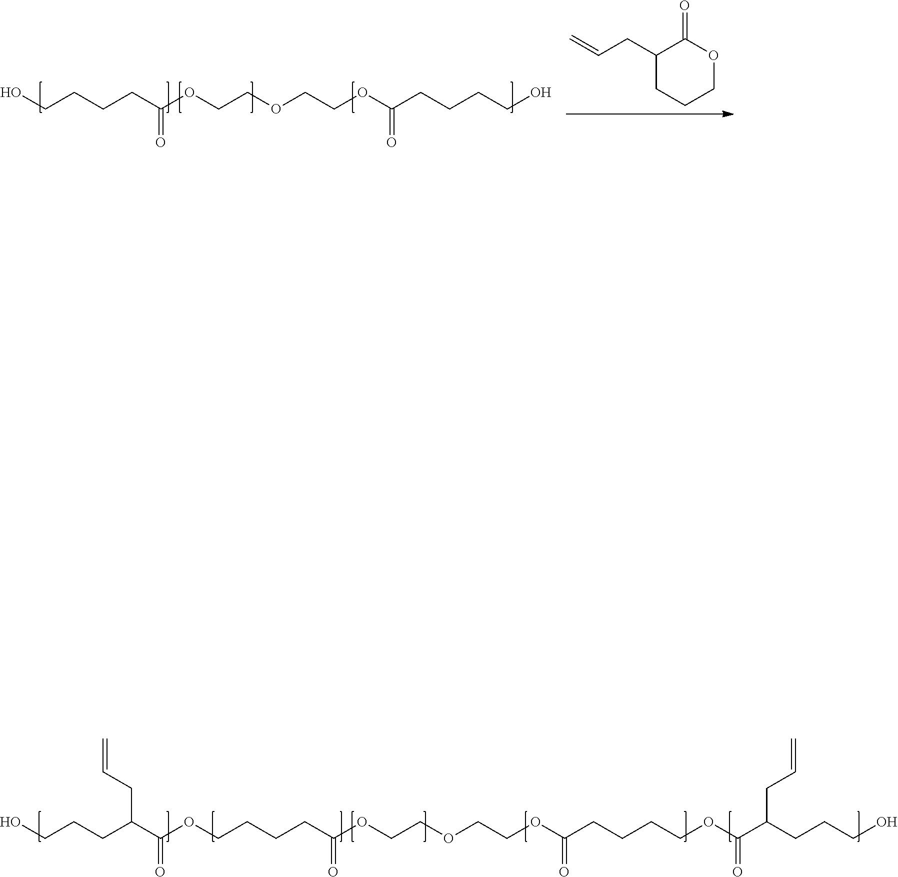

For block copolymerization, AVL (0.17 mL, 1.25 mmol, target repeating unit is 25) as second monomer was injected into the reactive mixture by cannulation, and the resulting mixture was further stirred at room temperature for 4 hrs. The as-synthesized polymer solution was precipitated from a mixture of ethyl ether and hexane (70/30 v/v) for purification, and residues were dried in a vacuum oven at room temperature overnight. Example 4—Synthesis of Poly(allylvalerolactone-valerolactone)-MPA With 10 Percent Functionalized Polymer

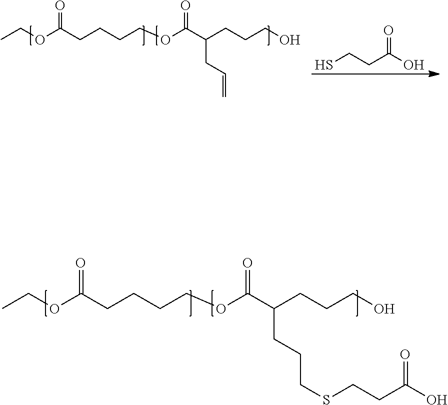

The poly(avl-vl) polymer comprising about 10% Allyl(valerolactone) prepared in Example 1 (1.63 g, 2.06 mmol allyl groups) and 3-mercaptopropionic acid (MPA) (1.0 ml, 10.3 mmol) were dissolved into 10 ml of anhydrous DMF in a 25 mL flame dried and argon purged round bottom flask. 2, 2-Dimethoxy-2-phenylacetophenone (264 mg, 1.03 mmol) was added into reaction flask. The system was sealed with a rubber septum and purged with argon for 30 minutes. Then the reaction solution was exposed to the UV-lamp (λ_{exc}=365 nm) for 2 hours.

The resulting solution was added dropwise into 100 ml of chilled methanol and chilled overnight in the freezer. The precipitate was rinsed with ice-cold methanol (100 ml×3) and collected by centrifuge. After overnight drying by vacuum, the final polymer product was made as a white waxy solid. Yield: 1.70 g/92.0%, (99.0% pure/NMR). ¹H NMR (500 MHz, CDCl₃/TMS, ppm) δ: 4.09 (m, —CH₂—O—), 3.68 (m, CH₃CH₂O—), 2.79 (t, —S—CH₂CH₂C(O)OH), 2.67 (t, —S—CH₂CH₂C(O)OH), 2.56 (t, —CH₂CH₂CH₂—S—), 2.34 (m, vl, —CH₂CH₂C(O)O—, avl, —CH—CH₂CH₂CH₂—S—), 1.69 (m, vl, —OC(O)CH₂CH₂CH₂—, avl, —OC(O)CHCH₂CH₂—), 1.27 (t, CH₃CH₂O—).

### Example 5—Synthesis of Poly(allylvalerolactone-valerolactone)-MPA With 20 Percent Functionalized Polymer

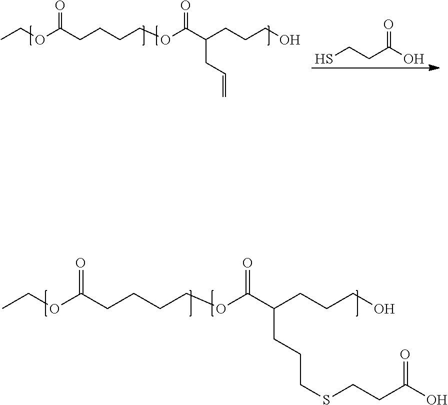

The poly(avl-vl) polymer comprising about 20% Allyl(valerolactone) prepared in Example 2 and 3-mercaptopropionic acid (MPA) (1.7 ml, 19.8 mmol) were dissolved into 10 ml of anhydrous DMF in a 25 mL flame dried and argon purged round bottom flask. 2, 2-Dimethoxy-2-phenylacetophenone (509 mg, 1.99 mmol) was added into reaction flask. The system was sealed with a rubber septum and purged with argon for 30 minutes. Then the reaction solution was exposed to the UV-lamp (λ_{exc}=365 nm) for 2 hours.

The resulting solution was added dropwise into 100 ml of chilled methanol and chilled overnight in the freezer. The precipitate was rinsed with ice-cold methanol (100 ml×3) and collected by centrifuge. After overnight drying by vacuum, the final polymer product was made as a white waxy solid. Yield: 2.07 g/(85.5%, (99.4% pure/NMR). ¹H NMR (500 MHz, CDCl₃/TMS, ppm) δ: 4.09 (m, —CH₂—O—), 3.66 (m, CH₃CH₂O—), 2.78 (t, —S—CH₂CH₂C(O)OH), 2.66 (t, —S—CH₂CH₂C(O)OH), 2.55 (t, —CH₂CH₂CH₂—S—), 2.36 (m, vl, —CH₂CH₂C(O)O—, avl, —CH—CH₂CH₂CH₂—S—), 1.68 (m, vl, —OC(O)CH₂CH₂CH₂—, avl, —OC(O)CHCH₂CH₂—), 1.27 (t, CH₃CH₂O—).

### Example 6—Synthesis of Poly(allylvalerolactone-valerolactone)-MPA-ALE with 10 Percent Functionalized Polymer

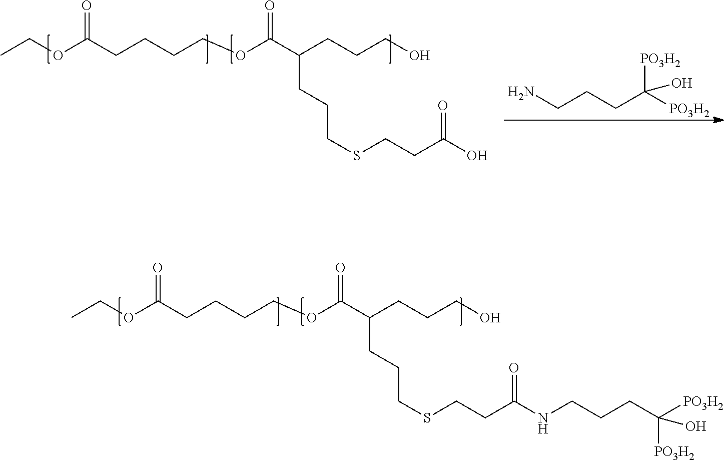

The poly(avl-vl)-MPA prepared in Example 3 (1.60 g, 2.02 mmol carboxylic groups) and N-hydroxysuccinimide (0.345 g, 3.03 mmol) were dissolved into 25 ml of anhydrous THF in a 50 mL flame dried and argon purged round bottom flask. N, N-Dicyclohexyl carbodiimide (0.66 g, 3.23 mmol) was added into the reaction solution. The reaction solution was stirred under argon for 6 hours.

Alendronate aqueous solution was prepared by dissolving alendronate sodium trihydrate (1.30 g, 4.0 mmol) and trimethylamine (5.0 ml, 35.8 mmol) into 50 ml of water and added to the above-solution. The side-product DCU was removed by centrifugation and washed with fresh anhydrous THF (10 ml×3). The supernatant was concentrated/dried by vacuum and then dissolved into 25 ml of anhydrous dioxane. The resulted dioxane solution was added dropwise into the previously prepared alendronate basic aqueous solution. The reaction system was stirred under argon for 24 hours. The reaction system was concentrated by rotavapor to remove most of dioxane, and then quenched with 0.5 M aq. HCl and titrated to pH around 2.0. The system was purified through dialysis against MWCO 2 kDa membrane, and dried by vacuum. The crude product was further purified by washing with methanol (25 ml×3). The final product was made as white waxy solid. Yield: 1.76 g/78.2%, (92.7% pure/NMR). ¹H NMR (500 MHz, DMSO-d₆/TMS, ppm) δ: 7.87 (b, —C(O)NH—), 4.09 (b, —OH), 3.99 (m, —CH₂—O—, CH₃CH₂O—), 3.35 (t, —CH₃—CH₂—OH), 2.96 (m, —C(O)NH—CH₂—), 2.63 (t, —S—CH₂CH₂C(O)OH), 2.46 (m, —S—CH₂CH₂C(O)OH, —CH₂CH₂CH₂—S—), 2.31 (m, vl, —CH₂CH₂C(O)O—, avl, —CH—CH₂CH₂CH₂—S—), 1.79 (m, —C(O)NH—CH₂—CH₂—), 1.69 (m, —C(O)NH—CH₂—CH₂—CH₂—), 1.56 (m, vl, —OC(O)CH₂CH₂CH₂—, avl, —OC(O)CHCH₂CH₂—), 1.16 (t, CH₃CH₂O—).

### Example 7—Synthesis of Poly(allylvalerolactone-valerolactone)-MPA-ALE with 20 Percent Functionalized Polymer

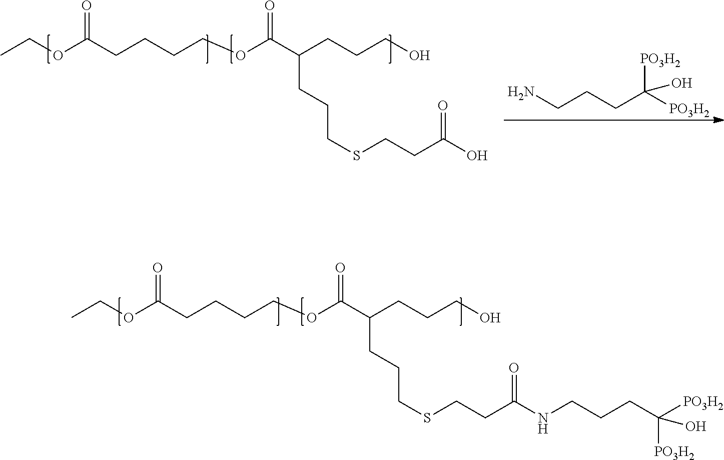

The poly(avl-vl)-MPA prepared in Example 4 (2.00 g, 3.97 mmol carboxylic groups) and N-hydroxysuccinimide (0.685 g, 5.96 mmol) were dissolved into 25 ml of anhydrous dioxane in a 50 mL flame dried and argon purged round bottom flask. N, N-Dicyclohexyl carbodiimide (1.31 g, 6.35 mmol) was added into the reaction solution. The reaction solution was stirred under argon for 6 hours.

Alendronate aqueous solution was prepared by dissolving alendronate sodium trihydrate (2.58 g, 7.94 mmol) and trimethylamine (6.0 ml, 43 mmol) into 60 ml of water and added to the above-solution.

The side-product DCU was removed by centrifugation and washed with 5 ml of fresh anhydrous dioxane. The dioxane solution (30 ml) was added dropwise into the previously prepared alendronate basic aqueous solution. The reaction system was stirred under argon for 24 hours. The reaction system was concentrated by rotavapor to remove most of dioxane, and then quenched with 0.5 M aq. HCl and titrated to pH around 2.0. The system was purified through dialysis against MWCO 2 kDa membrane, and dried by vacuum. The crude product was further purified by washing with methanol (25 ml×3). The final product is made as white waxy solid. Yield: 2.07 g (62.9%, (94.0% pure/NMR). ¹H NMR (500 MHz, DMSO-d₆/TMS, ppm) δ: 7.87 (b, —C(O)NH—), 4.14 (b, —OH), 3.99 (m, —CH₂—O—, CH₃CH₂O—), 3.35 (t, —CH₃—CH₂—OH), 2.97 (m, —C(O)NH—CH₂—), 2.63 (t, —S—CH₂CH₂C(O)OH), 2.46 (m, —S—CH₂CH₂C(O)OH, —CH₂CH₂CH₂—S—), 2.31 (m, vl, —CH₂CH₂C(O)O—, avl, —CH—CH₂CH₂CH₂—S—), 1.79 (m, —C(O)NH—CH₂—CH₂—), 1.70 (m, —C(O)NH—CH₂—CH₂—CH₂—), 1.55 (m, vl, —OC(O)—CH₂CH₂CH₂—, avl, —OC(O)—CHCH₂CH₂—), 1.17 (t, CH₃CH₂O—).

### Example 8—Preparation of Poly(allylglycidyl ether-glycidol) Polymers

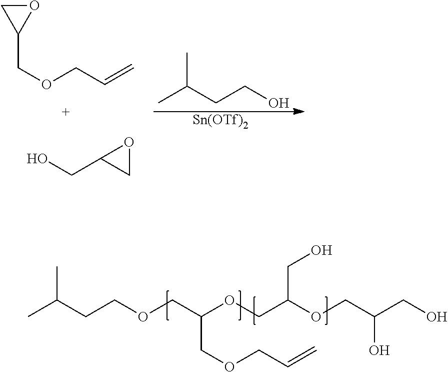

A flame dried 50 mL round bottom flask was equipped with a stir bar, sealed with rubber septum and argon purged for 10 minutes. Sn(OTf)₂ (46.0 mg, 0.11 mmol) was directly added into the flask. The reaction flask was capped with septum again and purged once more with argon for 10 minutes. Then 3-methyl-1-butanol (Isoamyl alcohol, IAOH) (340 mg, 3.86 mmol) was added into the reaction flask via micro syringe under argon to offset the created pressure. The initiator-catalyst mixture was then allowed to stir at room temperature for 30 minutes before lowering the reaction vessel into an ice/salt bath.

After the reaction vessel had been cooled for 10 minutes, the allyl glycidyl ether (AGE) (6.50 ml, 55.3 mmol) was added dropwise through syringe slowly to the stirring reaction in the ice/salt batch under argon. The glycidol (GLY) (15.0 ml, 225 mmol) was added dropwise through syringe very slowly to ensure the exothermic reaction did not overheat and decompose.

The reaction system was stirred under argon for 18 hours in the ice/water bath. The resulting polymer (solidified as a clear gel) was slowly warmed up to the room temperature, dissolved into 25 ml of methanol, and precipitated into vigorously stirring 1 L of ethyl acetate. The system was chilled overnight in the freezer before carefully decanting off the ethyl acetate. The residue was re-dissolved into methanol and precipitated/rinsed with ethyl acetate (100 ml×3). The product was collected by centrifuge and dried by vacuum for three days. The final polymer product was made as a translucent, viscous liquid. Yield: 20.67 g/89.8%, (95.4% pure/NMR). NMR data analysis shows the polymer molecular weight at 10.0 KDa and AGE content at 16.7% by molar. ¹H NMR (500 MHz, CDCl₃/TMS, ppm) δ: 5.90 (m, H₂C═CH—), 5.31 (m, H₂C═CH—), 4.02 (m, H₂C═CH—CH₂—O—), 4.02-3.48 (m, (CH₃)₂CHCH₂CH₂O—, AGE units 5H, GLY units 5H), 0.90 (d, (CH₃)₂CH—).

### Example 9—Synthesis of Poly(allylglycidyl ether-glycidol)-MPA Polymers

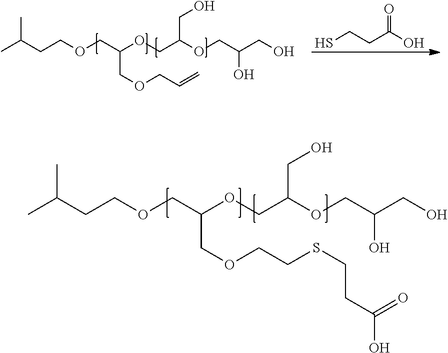

The poly(allylglycidyl ether-glycidol) copolymer prepared in Example 7 (13.67 g, 28.3 mmol vinyl groups) and 3-mercaptopropionic acid (MPA) (12.3 ml, 141.5 mmol) were dissolved into 60 ml of anhydrous DMF in a 250 mL flame dried and argon purged round bottom flask. 2, 2-Dimethoxy-2-phenylacetophenone (3.63 g, 14.2 mmol) was added into reaction flask. The system was sealed with a rubber septum and purged with argon for 30 minutes. Then the reaction solution was exposed to the UV-lamp (λ_{exc}=365 nm) for 2 hours.

The resulting solution was added dropwise into 500 ml of ethyl acetate and chilled overnight in the freezer. After carefully decanting off the ethyl acetate, the residue was re-dissolved into methanol and precipitated/rinsed with ethyl acetate (100 ml×3). The product was collected by centrifuge. After overnight dried by vacuum, the final polymer product was made as a translucent, viscous liquid. Yield: 16.06 g/96.3%, (91.2% pure/NMR). ¹H NMR (500 MHz, D₂O, ppm) δ: 3.90-3.46 (m, AGE units 7H, GLY units 5H, (CH₃)₂CHCH₂—O—), 2.72 (t, —S—CH₂CH₂C(O)OH), 2.60 (t, —S—CH₂CH₂C(O)OH), t, —OCH₂CH₂CH₂—S—), 1.79 (m, —OCH₂CH₂CH₂—S—), 0.80 (d, (CH₃)₂CH—).

### Example 10—Synthesis of Poly(allylglycidyl ether-glycidol)-MPA-ALE

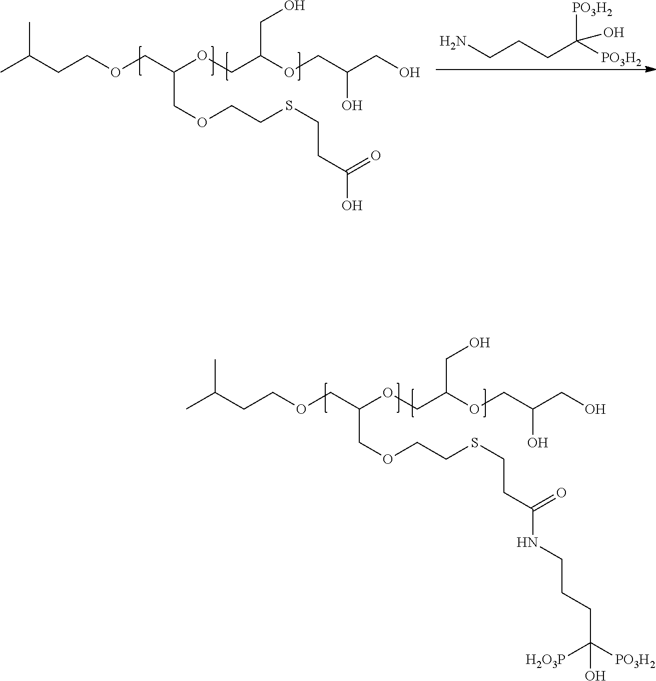

The poly(allylglycidyl ether-glycidol)-MPA copolymer prepared in Example 8 (16.06 g, 24.2 mmol carboxylic groups) and N-hydroxy succinimide (4.17 g, 36.3 mmol) were dissolved into 115 ml of anhydrous dioxane in a 250 mL flame dried and argon purged round bottom flask. N, N-dicyclohexyl carbodiimide (7.99 g, 38.7 mmol) was added into the reaction solution. The reaction solution was stirred under argon overnight.

Alendronate aqueous solution was prepared by dissolving alendronate sodium trihydrate (14.2 g, 43.6 mmol) and sodium carbonate (24.6 g, 232 mmol) into 280 ml of water and added to the above-solution.

The side-product DCU was removed by filtration and washed with 25 ml of fresh anhydrous dioxane. The dioxane solution (˜140 ml) was added dropwise into the previously prepared alendronate basic aqueous solution. The reaction system was stirred under argon for 24 hours. The reaction system was concentrated by Rota vapor to remove most of dioxane. The generated insoluble white floating solid was identified mainly as DCU side product and was removed by filtration. The solution was then quenched with 0.5 M aq. HCl and titrated to pH around 6-7. The system was purified through dialysis against MWCO 2 kDa membrane, and dried by vacuum. The final product is made as a translucent glassy solid/gel. Yield: 7.70 g/34.7%, (97.6% pure/NMR). ¹H NMR (500 MHz, D₂O, ppm) δ: 3.90-3.53 (m, AGE units 7H, GLY units 5H, (CH₃)₂CHCH₂—O—), 3.10 (m, —C(O)NH—CH₂—), 2.71 (t, —S—CH₂CH₂C(O)OH), 2.54 (t, —S—CH₂CH₂C(O)OH), 2.44 (t, —OCH₂CH₂CH₂—S—), 1.79 (m, —C(O)NH—CH₂—CH₂—, —OCH₂CH₂CH₂—S—), 1.70 (m, —C(O)NH—CH₂—CH₂—CH₂—), 0.80 (d, (CH₃)₂—CH—).

### Example 11—Synthesis of Linear, Phosphonate-Functionalized Polyglycidol

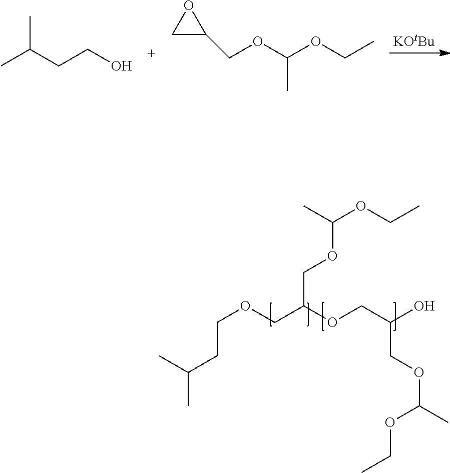

Isoamylalcohol (4.28 mmol) was dissolved in diglyme (15 mL) in a flask and potassium tert-butoxide (0.43 mL of a 1 M solution in THF, 0.43 mmol) was added. The resulting tert-butyl alcohol was removed by distillation. Glycidol acetal (15.0 g, 0.10 mol) was then added and allowed to stir for 20 h at 120° C. The solvent was removed by vacuum and a viscous liquid was acquired.

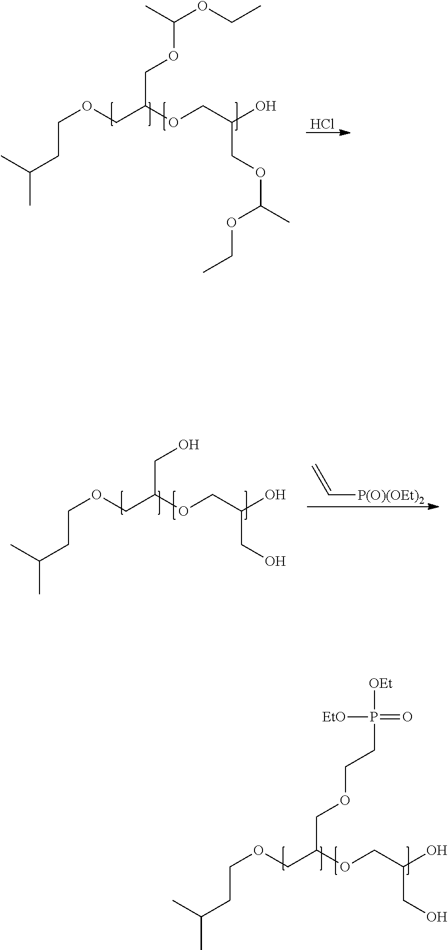

The poly(glycidol acetal) (1.0 g) was dissolved in tetrahydrofuran (120 mL) followed by addition of aqueous 32% HCl (6.1 g). After 5 hours, the polyglycidol was purified by precipitation in ethyl acetate or acetone and dried by vacuum.

Polyglycidol (0.579 g, 0.355 mmol) was dissolved in DMF (12 mL) and potassium tert-butoxide (0.35 mL of a 1 M solution in THF, 0.35 mmol) was added over 2 hours. The solution was then stirred at room temperature for 30 minutes. The tert-butanol that formed was removed by distillation. Diethyl vinylphosphonate (DEPE, 0.874 g, 5.328 mmol) was added, and the reaction mixture stirred for 144 h at room temperature. The product was removed by filtration and dried in vacuum. The product was re-dissolved in chloroform and precipitated in cold pentane.

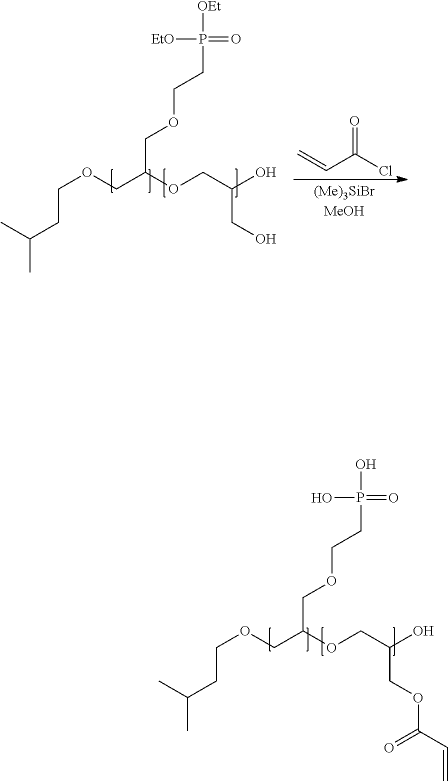

Next, the product (0.5996 g, 2.688 mmol OH) was dissolved in dry dichloromethane (20.0 mL) and acryloyl chloride (0.3159 g, 3.490 mmol, 1.3 eq.) was slowly added. The mixture was stirred at 60° C. for 17 h. Subsequently, trimethylsilyl bromide (0.7855 g, 5.517 mmol, 3.0 eq. with respect to DEPE groups) was added. The mixture refluxed with stirring for 5 hours. Ethanol (8.7 mL) was added and dichloromethane was removed by vacuum. The solution then stirred for 1 h at room temperature and stored at -20° C.

### Example 12—Synthesis of Semi-Branched or Hyper-Branched Phosphonate-Functionalized Polyglycidol

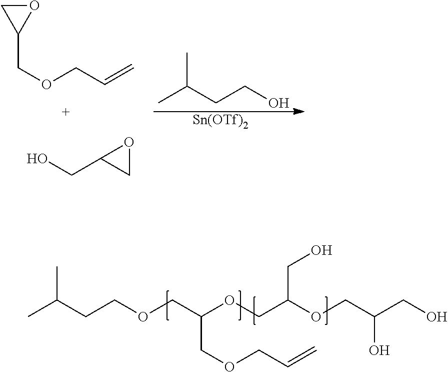

The copolymerization of glycidol and allyl glycidyl ether was performed neat under argon in ice/water bath overnight. 3-methyl-1-butanol (isoamyl alcohol, 3.33×10⁻⁴ mol, 0.066 eq) and tin(II) triflate (Sn(OTf)₂, 9.45×10⁻⁶ mol, 0.00035 eq) were added to a round bottom flask and stirred for 30 minutes. After the complexation of the initiator to the catalyst, the glycidol (1.44 g; 19.47 mmol; 4.0 eq) and allyl glycidyl ether (0.56 g; 4.87 mmol; 1.0 eq) were added dropwise. After stirring was completely impeded (reaction time varied with temperature), the crude viscous polymer product was dissolved in a minimal amount of methanol and precipitated into vigorously stirring acetone or ethyl acetate, which was then decanted to afford the pure GLY/AGE polymer product as translucent viscous material.

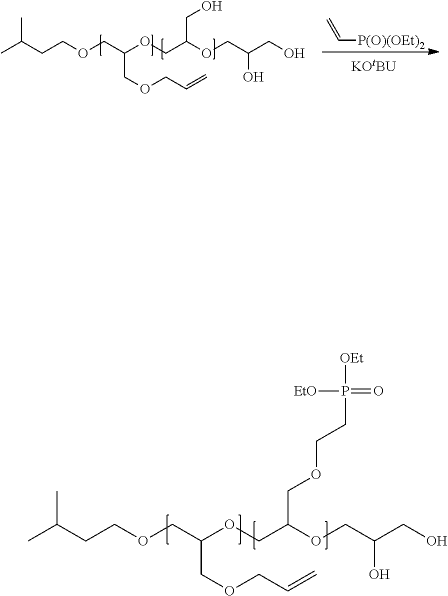

The resulting polymer (0.579 g) was dissolved in DMF (12 mL) and potassium tert-butoxide (0.35 mL of a 1 M solution in THF, 0.35 mmol) was added over 2 hours. The solution was then stirred at room temperature for 30 minutes. The tert-butanol that formed was removed by distillation. Diethyl vinylphosphonate (DEPE, 0.874 g, 5.328 mmol) was added, and the reaction mixture stirred for 144 h at room temperature. The product was removed by filtration and dried in vacuum. The product was redissolved in chloroform and precipitated in cold pentane.

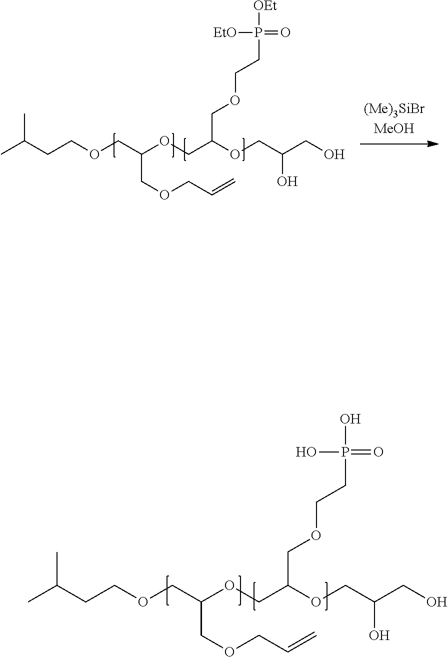

Next, the product (0.5996 g, 2.688 mmol OH) was dissolved in dry dichloromethane (20.0 mL). Subsequently, trimethylsilyl bromide (0.7855 g, 5.517 mmol, 3.0 eq. with respect to DEPE groups) was added. The mixture refluxed with stirring for 5 hours. Ethanol (8.7 mL) was added and dichloromethane was removed by vacuum. The solution then stirred for 1 h at room temperature and stored at −20° C.

### Example 13—Synthesis of Semi-Branched or Hyper-Branched Alendronate-Functionalized Polyglycidol

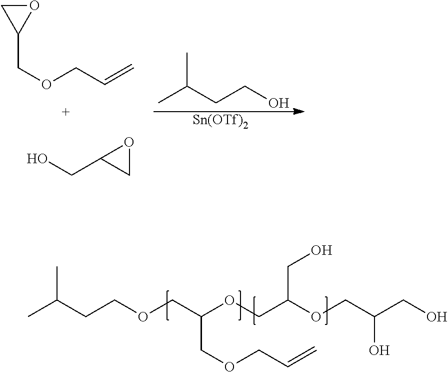

The copolymerization of glycidol and allyl glycidyl ether was performed neat under argon in ice/water bath overnight. 3-methyl-1-butanol (isoamyl alcohol, 3.33×10⁻⁴ mol, 0.066 eq) and tin(II) triflate (Sn(OTf)₂, 9.45×10⁻⁶ mol, 0.00035 eq) were added to a round bottom flask and stirred for 30 minutes. After the complexation of the initiator to the catalyst, the glycidol (1.44 g; 19.47 mmol; 4.0 eq) and allyl glycidyl ether (0.56 g; 4.87 mmol; 1.0 eq) were added dropwise. After stirring was completely impeded (reaction time varied with temperature), the crude viscous polymer product was dissolved in a minimal amount of methanol and precipitated into vigorously stirring acetone or ethyl acetate, which was then decanted to afford the pure GLY/AGE polymer product as translucent viscous material.

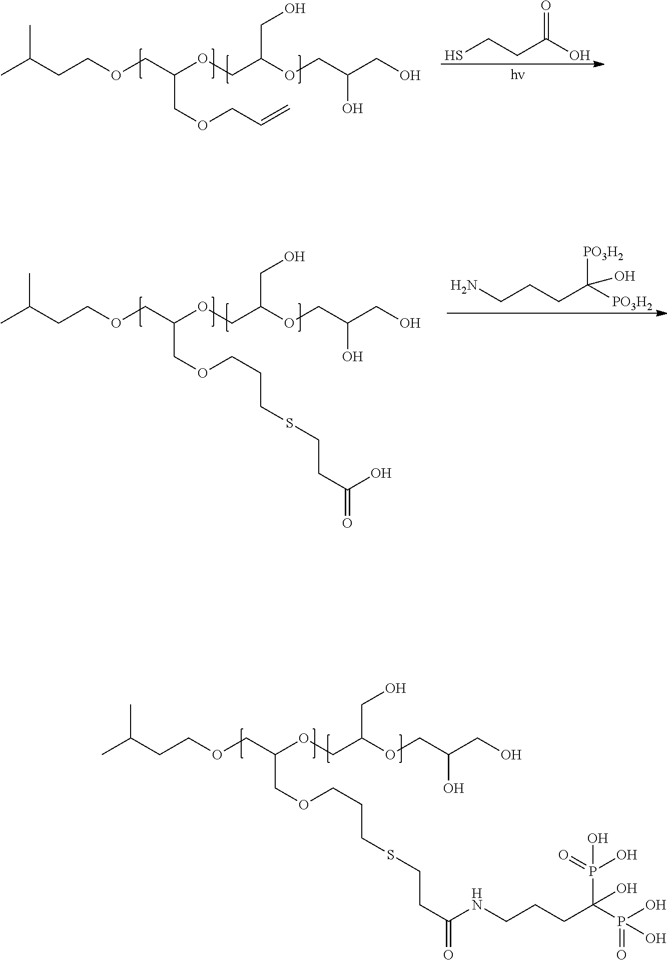

The allyl groups of the polymers were converted to carboxylic acid groups via thiol-ene chemistry with 3-mercaptopropionic acid. The carboxylic acid was then activated with NHS/DCC and reacted with alendronate in a basic aqueous/dioxane solvent system. The polymer was purified by dialysis and dried under vacuum.

### Example 14—Dip Coating

The poly(allylglycidyl ether-glycidol)-MPA-ALE of Example 9 is dissolved in ethyl acetate at a concentration of 2.0% (wt/wt) in a sterile vessel. Cefazolin is also dissolved in the ethyl acetate at a concentration of 2.0% (wt/wt).

A stainless steel plate for use as an orthopedic implant is sterilized and dipped, using tweezers, in the solution of polymer and cefazolin in ethyl acetate. The stainless steel plate is left in the solution for three seconds and removed using tweezers. The plate is allowed to drip for ten seconds to remove excess liquid ethyl acetate and is placed on a sterile rack for ten minutes to allow the remaining ethyl acetate to evaporate. The stainless steel plate is then irradiated with UV light to ensure that the surface is sterile. The plate is then used to set a broken bone.

### Example 15—Spray Coating

The poly(allylglycidyl ether-glycidol)-MPA-ALE of Example 9 is dissolved in N-methyl-2-pyrrolidone at a concentration of 2.0% (wt/wt) in a sterile manual spray bottle. Vancomycin is also dissolved in the N-methyl-2-pyrrolidone at a concentration of 2.0% (wt/wt).

A stainless steel plate for use as an orthopedic implant is sterilized and placed on a wire drying rack. The plate is sprayed by hand for twenty seconds with the solution of polymer and vancomycin in N-methyl-2-pyrrolidone. After the first ten seconds of spraying, the plate is turned over and the other side of the plate is sprayed. The plate is left on the drying rack for ten minutes to allow the remaining N-methyl-2-pyrrolidone to evaporate. The stainless steel plate is then irradiated with UV light to ensure that the surface is sterile. The plate is then used to set a broken bone.

### Example 16—Characterization of Coatings

The poly(allylglycidyl ether-glycidol)-MPA-ALE of Example 9 is dissolved in ethyl acetate at a concentration of 1.0% (wt/wt). Using a pipette, the solution of polymer in ethyl acetate is added to a 1-cm² titanium surface (about 2-3 drops of solution are used). Enough of the solution is used to cover the entire surface. The ethyl acetate is allowed to evaporate over ten minutes.

The surface is analyzed using scanning electron microscopy (SEM). The mass of the titanium surface prior to treatment with the polymer coating solution is 1.000000 g. After treatment, the mass of the titanium surface is 1.000001 g. Accordingly, the mass of the polymer coating is calculated as 1.0×10⁻⁶ g. The coating density is calculated as 1.0×10⁻⁶ g/cm².

### Example 17—Durability of the Coatings

The polymer-coated titanium surface of Example 12 is affixed to a solid surface. A 10-cm radius rubber wheel covered in a latex coating is spun using a motor at a rate of 100 rpm. The spinning wheel is pressed against the titanium surface at a pressure of 1.0 psi for one minute. Afterwards the titanium surface is re-weighed. The subsequent weight of the titanium surface is 1.0000005 g. Accordingly, the mass of the polymer coating is calculated as 0.5×10⁻⁶ g, and the coating density is calculated as 0.5×10⁻⁶ g/cm² after treating with the rubber wheel. The titanium surface is extracted three times with ethyl acetate. The combined ethyl acetate layers are combined and evaporated. The mass of the residue is 0.5×10⁻⁶ g.

### EQUIVALENTS

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

1. A polymeric coating for a substrate, the coating comprising:
a polymeric backbone;
a substrate-coordinating group; and
a reactive functional group.

2. The polymeric coating of claim 1, wherein the polymeric backbone comprises a polyglycidol.

3. The polymeric coating of claim 1, wherein the polymeric backbone comprises a polyester.

4. The polymeric coating of claim 3, wherein the polyester is a polyvalerolactone.

5. The polymeric coating of claim 2, wherein the polyglycidol backbone comprises a polyallyl glycidyl ether-polyglycidol copolymer.

6. The polymeric coating of claim 2, wherein the polyglycidol backbone is linear, branched, or hyperbranched.

7. The polymeric coating of claim 2, wherein the polyglycidol backbone is branched.

8. The polymeric coating of claim 4, wherein the polyvalerolactone backbone comprises a polyallylvalerolactone-polyvalerolactone copolymer.

9. The polymeric coating of claim 1, wherein the substrate-coordinating group is a metal-coordinating group.

10. The polymeric coating of claim 9, wherein the metal-coordinating group is a monophosphonate group, a bisphosphonate group, a diol, or a catechol.

11. The polymeric coating of claim 10, wherein the bisphosphonate group is selected from the group consisting of: alendronate; risendronate; etidronate; clodronate; tiludronate; pamidronate; neridronate; olpadronate; ibandronate; and zoledronate.

12. The polymeric coating of claim 10, wherein the diol is selected from the group consisting of: ethylene glycol; and propylene glycol.

13. The polymeric coating of claim 1, wherein the reactive functional group is an alkene, an alkyne, or an epoxide.

14. The polymeric coating of claim 1, wherein the coating further comprises a binding agent.

15. The polymeric coating of claim 1, comprising a polymer of Formula I: 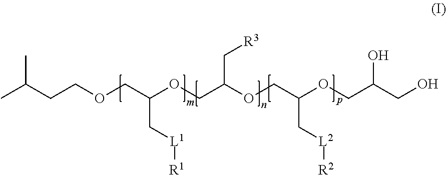
wherein:
L¹ is independently, at each occurrence, —(CR^{1A}R^{1B})_{q}—, —O(CR^{1A}R^{1B})_{q}—, —(CR^{1A}R^{1B})O_{q}—, —(CR^{1A}R^{1B})C(O)O_{q}— or —OC(O)(CR^{1A}R^{1B})_{q}—;
L² is independently, at each occurrence:
—(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;
—O(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;
—C(O)(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—; or
—OC(O)(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;
R¹ is independently, at each occurrence, —C₁-C₆ alkyl, —C₁-C₆ alkenyl, —C₁-C₆ alkynyl, —CH(O)CH₂, or —OH, wherein each alkyl, alkenyl, or alkynyl is optionally substituted with a drug, a conjugating group, a -PEG₁₋₆ bonded drug, or a -PEG₁₋₆ bonded conjugating group;
R^{1A} and R^{1B} are each independently, at each occurrence, —H —OH, or —NH₂;
R² is independently, at each occurrence, —CR^{2C}R^{2D}P(O)(OH)₂; —CR^{2C}(P(O)(OH)₂)₂; —C(P(O)(OH₂)₃, —CO₂H, —C₆(R^{2A})₃(OH)₂, or —CR^{2A}R^{2B}CR^{2B}R^{2C}R^{2D};
R^{2A} and R^{2B} are each independently, at each occurrence, —H —OH, or —NH₂;
R^{2C} and R^{2D} are each independently, at each occurrence, —H, —OH or —NH₂;
R³ is independently, at each occurrence, —C₁-C₆ alkenyl, —C₁-C₆ alkynyl, —C(O)C₁-C₆ alkyl, —C(O)C₁-C₆ alkenyl, —C(O)C₁-C₆ alkynyl, —OH, —OC₁-C₆ alkyl, —OC₁-C₆ alkenyl, —OC₁-C₆ alkynyl, —OC(O)C₁-C₆ alkyl, —OC(O)C₁-C₆ alkenyl, —OC(O)C₁-C₆ alkynyl, wherein each alkyl, alkenyl, or alkynyl is optionally substituted with a drug, a conjugating group, a -PEG₁₋₆ bonded drug, or a -PEG₁₋₆ bonded conjugating group;
m is independently an integer between 1 and 10,000;
n is independently an integer between 1 and 10,000;
p is independently an integer between 1 and 10,000;
q is independently, at each occurrence, an integer between 0 and 6; and
t is independently, at each occurrence, 0 or 1.

16-27. (canceled)

28. The polymeric coating of claim 1, comprising a polymer of Formula II: 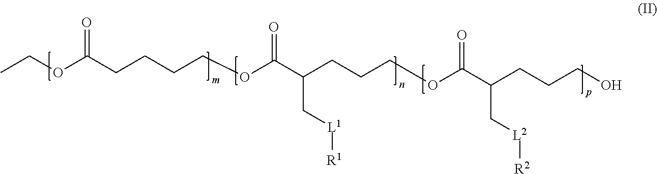
wherein:
L¹ is independently, at each occurrence, —(CR^{1A}R^{1B})_{q}—, —O(CR^{1A}R^{1B})_{q}—, —(CR^{1A}R^{1B})O_{q}—, —(CR^{1A}R^{1B})C(O)O_{q}—, —(CR^{1A}R^{1B})OC(O)_{q}—, or —OC(O)(CR^{1A}R^{1B})_{q}—;
L² is independently, at each occurrence:
—(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;
—O(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;
—C(O)(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—; or
—OC(O)(CR^{2A}R^{2B})_{q}—(S)ₜ(CR^{2A}R^{2B})_{q}—(C(O))ₜ(CR^{2A}R^{2B})_{q}—(NH)ₜ(CR^{2A}R^{2B})_{q}—;
R¹ is independently, at each occurrence, —C₁-C₆ alkyl, —C₁-C₆ alkenyl, —C₁-C₆ alkynyl, —CH(O)CH₂, or —OH, wherein each alkyl, alkenyl, or alkynyl is optionally substituted with a drug, a conjugating group, a -PEG₁₋₆ bonded drug, or a -PEG₁₋₆ bonded conjugating group;
R^{1A} and R^{1B} are each independently, at each occurrence, —H —OH, or —NH₂;
R² is independently, at each occurrence, —CR^{2C}R^{2D}P(O)(OH)₂; —CR^{2C}(P(O)(OH)₂)₂; —C(P(O)(OH₂)₃, —CO₂H, —C₆(R^{2A})₃(OH)₂, or —CR^{2A}R^{2B}CR^{2B}R^{2C}R^{2D};
R^{2A} and R^{2B} are each independently, at each occurrence, —H —OH, or —NH₂;
R^{2C} and R^{2D} are each independently, at each occurrence, —H, —OH or —NH₂;
m is independently an integer between 1 and 10,000;
n is independently an integer between 1 and 10,000;
p is independently an integer between 1 and 10,000;
q is independently, at each occurrence, an integer between 0 and 6; and
t is independently, at each occurrence, 0 or 1.

29-41. (canceled)

42. A method of preparing a polymeric coating for a substrate, the method comprising:
(i) preparing a polymeric backbone;
(ii) functionalizing the polymeric backbone with a substrate-coordinating group to create a functionalized polymer; and
(iii) contacting the substrate with the functionalized polymer.

43. (canceled)

44. A coated substrate, wherein the coating comprises:
a polymeric backbone;
a substrate-coordinating group; and
a reactive functional group.

45-59. (canceled)

60. A method of preparing a polymeric coating for a substrate, the method comprising:
(i) attaching an initiator to the surface of the substrate; and
(ii) polymerizing a polymer backbone from the attached initiator.

61-62. (canceled)
